(19) United States
(12) Patent Application Publication
(10) Pub. No.: US 2026/0091143 A1
(43) Pub. Date: Apr. 2, 2026

(54) NEAR-INFRARED IMAGING AGENT AND USES THEREOF

(71) Applicant: CASE WESTERN RESERVE UNIVERSITY, Cleveland, OH (US)

(72) Inventors: Susann Brady-Kalnay, Wellington, OH (US); Mette Johansen, Cleveland, OH (US)

(21) Appl. No.: 19/113,126

(22) PCT Filed: Sep. 20, 2023
(86) PCT No.: PCT/US2023/033289

§ 371 Date: Mar. 19, 2025

Related U.S. Application Data
(60) Provisional application No. 63/376,377, filed on Sep. 20, 2022.

(51) Int. Cl. A61K 49/00

(52) U.S. Cl. CPC A61K 49/0056; A61K 49/0034

(57) ABSTRACT

A near-infrared imaging agent includes a targeting peptide that specifically binds to and/or complexes with a proteolytically cleaved extracellular fragment of an immunoglobulin (Ig) superfamily cell adhesion molecule that is expressed by a cancer cell or another cell in the cancer cell microenvironment, an optional spacer directly linked to the targeting peptide; and a near-infrared fluorophore that is directly or indirectly linked to the targeting peptide or optional spacer via a natural or non-natural linkage.

### RELATED APPLICATION

This application claims priority from U.S. Provisional Application No. 63/376,377, filed Sep. 20, 2022, the subject matter of which is incorporated herein by reference in its entirety.

### GOVERNMENT FUNDING

This invention was made with government support under CA217956 awarded by The National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Sep. 20, 2023, is named CWR-031969 st.26 and is 63,464 bytes in size.

### BACKGROUND

Glioblastoma multiforme (GBM) is the most common malignant adult astrocytoma, and has the lowest survival rates of malignant brain cancers. The prognosis for GBM is extremely poor, with a median survival of 12-15 months. Several biological characteristics contribute to the lethality of GBM tumors, including their uncontrolled proliferation in the restricted cranial space, their angiogenic nature and their extensive dispersion throughout the brain. Surgical resection remains the first line of treatment. While near complete resection of the gadolinium-enhancing part of the tumor improves survival, it is widely recognized that microscopic invading cells (“fingers” or “tentacles”) remain locally within 2-3 cm of the original tumor and can cause up to a 90% recurrence rate in patients. Magnetic resonance imaging (MRI) is currently used to delineate the tumor border prior to resection. Unfortunately, this is an imperfect tool, as the contrast enhancement agent, gadolinium, has variable enhancement since it is simply a marker of leaky vessels and does not visualize GBM cells that migrate away from the main tumor mass. Neurosurgeons identify tumor using their naked eye and their own judgment. The development of specific molecular imaging agents will allow detection of these tentacles invading cells during surgery to improve the extent of resection.

### SUMMARY

Embodiments described herein relate to near-infrared imaging agents for use in detecting, monitoring, and/or imaging cancer cells and/or cancer cell metastasis, migration, dispersal, and/or invasion in a subject, methods of detecting, monitoring, and/or imaging cancer cells and/or cancer cell metastasis, migration, dispersal, and/or invasion in a subject, methods of determining and/or monitoring the efficacy of a cancer therapeutic and/or cancer therapy administered to a subject in need thereof, methods of determining, monitoring, and/or imaging efficacy of surgical resection of cancer cells in a subject, and/or methods of treating cancer in a subject in need thereof.

In some embodiments, the near-infrared imaging agent can include a targeting peptide that specifically binds to and/or complexes with a proteolytically cleaved extracellular fragment of an immunoglobulin (Ig) superfamily cell adhesion molecule that is expressed by a cancer cell or another cell in the cancer cell microenvironment, an optional spacer directly linked to the targeting peptide, and a near-infrared fluorophore that is directly or indirectly linked to the targeting peptide or optional spacer via a natural or non-natural linkage.

In some embodiments, the near-infrared imaging agent administered to a subject has a signal to background ratio (SBR) upon fluorescent imaging effective to delineate the cancer cell or another cell in the cancer cell microenvironment from surrounding tissue. In some embodiments, the near-infrared fluorophore is hydrophobic or

lipophilic.

In other embodiments, the natural or non-natural linkage is not susceptible to proteolytic cleavage.

In some embodiments, the non-natural linkage includes an amide that links the targeting peptide or optional spacer to the near-infrared fluorophore.

In some embodiments, the near-infrared imaging agent can have the formula:

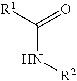

or a pharmaceutically acceptable salt thereof;
wherein R¹ is the near-infrared fluorophore; and
R² includes the targeting peptide and optional spacer.

In some embodiments, the near-infrared fluorophore includes at least one of a cyanine near-infrared fluorophore having a fluorescence in the first near infrared region or second near infrared region.

In some embodiments, the cyanine near-infrared fluorophore is a heptamethine cyanine near-infrared fluorophore. For example, the near-infrared fluorophore includes at least one of indocyanine green (ICG) or ICG-Osu.

In other embodiments, the near-infrared fluorophore can have formula:

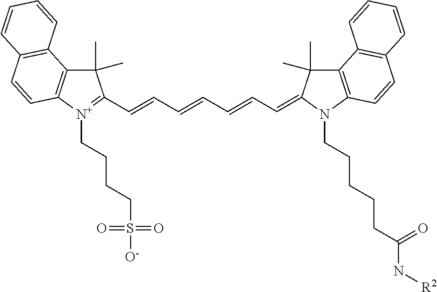

or a pharmaceutically acceptable salt thereof;
wherein R² includes the targeting peptide and optional spacer.

In some embodiments, the near-infrared fluorophore is directly linked to the targeting peptide via the non-natural linkage.

In other embodiments, the near-infrared fluorophore is directly linked to the spacer via the non-natural linkage.

In some embodiments, the spacer has a length and structure effective to at least maintain or preserve binding affinity of the linked targeting peptide to the proteolytically cleaved extracellular fragment and activity of the near-infrared fluorophore.

In some embodiments, the spacer can include natural and/or non-natural amino acids. For example, the spacer can include at least 3 natural or non-natural amino acids.

In some embodiments, the spacer has a length of 3, 4, 5, 6, 7, 8, 9, 10, 11, 12, 13, 14, 15, 16, 17, 18, 19, 20, 21, 22, 23, 24, 25, 26, 27, 28, 29, or 30 natural or non-natural amino acids.

In some embodiments, the spacer includes at least about 50%, at least about 60%, at least about 70%, at least about 80%, or at least about 90% glycine and/or serine residues.

In some embodiments, the spacer is a polyglycine or glycine/serine spacer. For example, the spacer can include the amino acid sequence of at least one of (GS)a, (GGS)b, or (GGGS)c, or (GGGGS)d and wherein a, b, c, and d are each independently 2, 3, 4, 5, or 6.

In some embodiments, the near-infrared imaging agent can be used in detecting, monitoring, and/or imaging cancer cells and/or cancer cell metastasis, migration, dispersal, and/or invasion, and/or for treating cancer in a subject.

In some embodiments, the near-infrared imaging agent can be configured for in vivo administration to a subject or ex vivo administration to biological sample of the subject.

Other embodiments described herein relate to a method of detecting cancer cells and/or cancer cell metastasis, migration, dispersal, and/or invasion in a subject in need thereof. The method can include administering to the subject an amount of the near-infrared imaging agent described herein. The agent bound to and/or complexed with the cancer cells can be detected to determine the location and/or distribution of the cancer cells in the subject.

In some embodiments, the cancer cells include at least one of a glioma, lung cancer, melanoma, breast cancer, or prostate cancer cells.

In other embodiments, the agent can be administered systemically, locally, or topically to the subject.

In some embodiments, the agent can be detected to define a tumor margin in a subject.

Other embodiments, relate to the use of the near-infrared imaging agent in fluorescent image-guided surgery.

Still other embodiments relate to the use of the near-infrared imaging agent in the preparation of a medicament for fluorescent image-guided surgery.

Other embodiments relate to a method of treating cancer in a subject in need thereof. The method includes administering to the subject a therapeutically effective amount of the near-infrared imaging agent as described herein. The agent bound to and/or complexed with the cancer cells can be irradiated at a wavelength effective to ablate the cancer cells.

In some embodiments, the cancer cells include at least one of a glioma, lung cancer, melanoma, breast cancer, or prostate cancer cells.

In other embodiments, the agent can be administered systemically, locally, or topically to the subject.

Still other embodiments relate to the use of the near-infrared imaging agent in photodynamic therapy or photothermal therapy.

Other embodiments relate to a compound of formula:

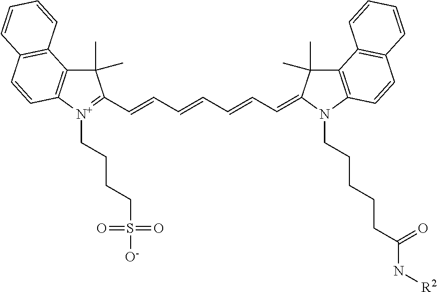

or a pharmaceutically acceptable salt thereof;
wherein R² includes a targeting peptide that specifically binds to and/or complexes with a proteolytically cleaved extracellular fragment of an immunoglobulin (lg) superfamily cell adhesion molecule that is expressed by a cancer cell or another cell in the cancer cell microenvironment and an optional spacer directly linked to the targeting peptide.

In some embodiments, R² consists of the targeting peptide.

In other embodiments, R² consists of the targeting peptide linked to a spacer and wherein the spacer separates the amide from the targeting peptide.

In some embodiments, the spacer has a length and structure effective to at least maintain or preserve binding affinity of the linked targeting peptide to the proteolytically cleaved extracellular fragment.

In some embodiments, the spacer includes natural and/or non-natural amino acids.

In other embodiments, the spacer can include at least 3 natural or non-natural amino acids.

In some embodiments, the spacer has a length of 3, 4, 5, 6, 7, 8, 9, 10, 11, 12, 13, 14, 15, 16, 17, 18, 19, 20, 21, 22, 23, 24, 25, 26, 27, 28, 29, or 30 natural or non-natural amino acids.

In some embodiments, the spacer includes at least about 50%, at least about 60%, at least about 70%, at least about 80%, or at least about 90% glycine and/or serine residues.

In some embodiments, the spacer is a polyglycine or glycine/serine spacer. For example, the spacer can include the amino acid sequence of at least one of (GS)a, (GGS)b, or (GGGS)c, or (GGGGS)d and wherein a, b, c, and d are each independently 2, 3, 4, 5, or 6.

### BRIEF DESCRIPTION OF THE DRAWINGS

FIGS. 1(A-B) illustrate in vivo tumor labeling of LN229 flank tumors with ICG-conjugated peptides over time. A. Flank tumor-bearing mice were injected with 300 nmol/kg of Scram-CLE-ICG, SBK2-CLE-ICG, Scram-ICG, and SBK2-ICG. Fluorescent images were acquired at the indicated times and are shown alongside baseline (BL) images acquired prior to the start of the experiment for a representative set of animals. B. Average radiant efficiencies (mean±SEM) of the in vivo tumor signal in each cohort of animals are plotted over time.

FIGS. 2(A-B) illustrate in vivo tumor signal at 24 h after injection of ICG-conjugated peptides. A. LN229 flank tumor-bearing mice 24 h following injection of Scram-CLE-ICG, SBK2-CLE-ICG, Scram-ICG, and SBK2-ICG at 300 nmol/kg. Top row shows photos with the tumor region outlined and bottom row shows the corresponding fluorescent images. Mice are those shown in FIG. 1 but with the scale optimized for the 24 h timepoint. B. Average radiant efficiency (mean±SEM) of the 24 h in vivo tumor signal in mice administered the indicated agents at 300 nmol/kg or 400 nmol/kg.

FIGS. 3(A-B) illustrate ex vivo fluorescent tumor signal detected 24 h following administration of ICG-conjugated peptides at 300 nmol/kg and 400 nmol/kg. A. Fluorescent images of excised tumors in mice treated with 300 nmol/kg of each agent. Representative tumors are shown for the number of mice indicated. B. Average radiant efficiencies (mean±SEM) of the 24 h ex vivo tumor signal in mice administered the indicated agents at 300 nmol/kg or 400 nmol/kg. Measurements were made on the excised tumors from the same mice used in FIG. 2.

FIGS. 4(A-B) illustrate in vivo tumor labeling with IRDye800-conjugated and TF8WS-conjugated peptides over time. A. Mice were injected with 400 nmol/kg Scram-CCLE-IR800 or SBK2-CCLE-IR800, and images were acquired every ten minutes for 60 minutes. Average radiant efficiency values of the tumor region for the two sets of mice (mean±SEM) were plotted. No significant differences were detected at any time. B. Mice were injected with 400 nmol/kg Scram-TF8WS or 400 nmol/kg SBK2-TF8WS and images were acquired at the indicated times. No significant differences were observed between the two groups of mice at any time. Inset plot shows data obtained for the TF8WS-conjugated peptides for the first 60 min plotted alongside the IR800-conjugated peptide data shown in panel A.

FIGS. 5(A-B) illustrate in vivo tumor signal at 60 min with IRDye800-conjugated and at 24 h with TF8WS-conjugated peptides. A. LN229 flank tumor-bearing mice following injection of the indicated agents at 400 nmol/kg at either 60 min (IRDye800-conjugated peptides) or 24 h (TF8WS-conjugated peptides). Top row shows photos with the tumor region outlined in each mouse. Bottom row shows fluorescent images superimposed on the photographic images. For each agent, a representative mouse for the cohorts plotted in FIG. 4 is shown. B. in vivo tumor fluorescence (mean±SEM) at 60 min (IRDye800-conjugated peptides) or 24 h (TF8WS-conjugated peptides) for the indicated agents. No significant differences were detected between Scram-CCLE-IR800 and SBK2-CCLE-IR800, or between Scram-TF8WS and SBK2-TF8WS.

FIGS. 6(A-B) illustrate ex vivo fluorescent tumor signals in mice treated with 400 nmol/kg IRDye800-conjugated peptides at 60 min or with 400 nmol/kg TF8WS-conjugated peptides at 24 h. A. Fluorescent images acquired in excised tumors from mice at 60 min or 24 h after injection of the indicated agents at 400 nmol/kg. Tumors shown are from representative mice. B. Average radiant efficiencies (mean±SEM) of the 24 h ex vivo tumor signal in mice administered the indicated agents at 400 nmol/kg. No significant differences were found between either pair of agents.

FIG. 7 illustrates molecular structures of the functionalized near-infrared fluorophores used to generate the PTPμ-targeted and control peptide-labeled agents. (Top) ICG-Osu contains an N-Hydroxysuccinimide (NHS) ester and was conjugated to the primary amine of the SBK2 or Scrambled peptides while the peptide was still attached to the resin. The manufacturer's stated molecular weight (MW) and the optimal excitation and emission wavelengths for fluorescence are provided. Analytical RP-HPLC chromatograms obtained at 220 nm for SBK2-CLE-ICG and SBK2-ICG are shown in the upper right corner. The agents are eluted at higher percentages of acetonitrile with SBK2-CLE-ICG exhibiting a retention time (RT) approximately 1.3 min shorter than that of SBK2-ICG. The amide bond between the fluorophore and peptide is shown as an inset in the chromatogram. (Middle) An additional cysteine was added to the N-terminus of SBK2-CLE and Scram-CLE peptides to allow conjugation of the hydrophilic IRDye 800CW maleimide in aqueous solution. The MW and wavelengths associated with optimal fluorescence as determined by the manufacturer are indicated. The chromatogram shows elution of the expected SBK2-CCLE-IR800 molecule with an intact succinimide moiety at 25.4 min. An asterisk (*) highlights an earlier peak representing the SBK2-CCLE-IR800 with an open succinimide ring eluting at 24.4 min. The open and closed succinimide ring linkages between fluorophore and peptides are inset. (Bottom) Representation of the acid form of the proprietary structure of the Tide Fluor 8WS fluorophore along with the MW and the manufacturer's stated wavelengths for optimal fluorescence. The TF8WS acid was conjugated to the N-terminal amine of the peptides on resin during the final step of synthesis. An analytical RP-HPLC chromatogram shows elution of the SBK2-TF8WS agent at 25.5 min with the amide bond linkage between the fluorophore and peptide (inset).

FIGS. 8(A-B) illustrate ex vivo fluorescence in kidneys, spleen and liver 24 h after administration of Scram-CLE-ICG, SBK2-CLE-ICG, Scram-ICG, and SBK2-ICG at 300 nmol/kg or ICG only at 400 nmol/kg. A. Fluorescent detected at Ex 745 nm/Em 820 nm in excised organs from mice injected with the indicated ICG-conjugated peptides at 300 nmol/kg. Representative organs are shown from the mice used in FIGS. 1 and 2. B. For each agent at 300 nmol/kg and for ICG only at 400 nmol/kg, the average radiant efficiency detected in different organs was compared and plotted. For kidney, the right and left kidney signals from each mouse were averaged and used as the kidney signal for that animal. Unpaired t-tests with Welch's correction were used to compare the fluorescent signals and the p-values obtained are summarized in Table 5.

FIGS. 9(A-B) illustrate ex vivo fluorescence in kidney, spleen and liver after injection of IR800-conjugated peptides or TF8WS-conjugated peptides at 400 nmol/kg. A. Fluorescence detected at Ex 745 nm/Em 800 nm in excised kidneys, spleen and liver from animals treated with Scram-CCLE-IR800 (top left) and SBK2-CCLE-IR800 (top right) for 60 min, and from animals treated with Scram-TF8WS (bottom left) and SBK2-TF8WS (bottom right) for 24 h. (a=right kidney, b=left kidney, c=spleen, d=liver). Representative organs are shown for each cohort. B. Plots of average radiant efficiencies measured in kidney, spleen and liver. For kidney, the right and left kidney signals from each mouse were averaged and used as the kidney signal for that animal. Significantly more fluorescence was detected in the kidneys and spleens of animals dosed with Scram-CCLE-IR800 compared to SBK2-CCLE-IR800 after 60 min. For kidneys, p<0.005, and for spleen, p<0.01. No significant difference in liver fluorescence was detected in mice treated with Scram-CCLE-IR800 compared to SBK2-CCLE-IR800. In mice treated with Scram-TF8WS and SBK2-TF8WS for 24 h, no significant differences in average radiant efficiency were measured in kidney, spleen, or liver.

FIGS. 10(A-B) illustrate ventral images of mice treated with PTPμ-targeted and control near-infrared imaging agents at various times to illustrate the biodistribution of the agents. A. Mice were imaged 60 min following injection of ICG-conjugated peptides at 300 nmol/kg (top row, Ex 745 nm/Em 820 nm) or IR800-conjugated and TF8WS-conjugated peptides at 400 nmol/kg (bottom row, Ex 745 nm/Em 800 nm) to document modes of agent clearance. B. Mice shown in A at 24 h following injection with ICG-conjugated peptides at 300 nmol/kg (top row) or TF8WS-conjugated peptides at 400 nmol/kg (bottom row). Mice injected with the IR800-conjugated peptides were sacrificed after acquiring images at 60 min so no 24 h images were obtained.

### DETAILED DESCRIPTION

Methods involving conventional molecular biology techniques are described herein. Such techniques are generally known in the art and are described in detail in methodology treatises, such as Current Protocols in Molecular Biology, ed. Ausubel et al., Greene Publishing and Wiley-Interscience, New York, 1992 (with periodic updates). Unless otherwise defined, all technical terms used herein have the same meaning as commonly understood by one of ordinary skill in the art to which the application pertains. Commonly understood definitions of molecular biology terms can be found in, for example, Rieger et al., Glossary of Genetics: Classical and Molecular, 5th Edition, Springer-Verlag: New York, 1991, and Lewin, Genes V, Oxford University Press: New York, 1994.

The articles “a” and “an” are used herein to refer to one or to more than one (i.e., to at least one) of the grammatical object of the article. By way of example, “an element” means one element or more than one element.

The term “log P” or “log P” refers to the log (base 10) of the n-octanol/water partition coefficient (log P_{ow}).

The terms “comprise,” “comprising,” “include,” “including,” “have,” and “having” are used in the inclusive, open sense, meaning that additional elements may be included. The terms “such as”, “e.g.,”, as used herein are non-limiting and are for illustrative purposes only. “Including” and “including but not limited to” are used interchangeably.

The term “or” as used herein should be understood to mean “and/or”, unless the context clearly indicates otherwise.

The term “agent” is used herein to denote a chemical compound, a mixture of chemical compounds, a biological macromolecule, or an extract made from biological materials.

The terms “cancer” or “tumor” refer to any neoplastic growth in a subject, including an initial tumor and any metastases. The cancer can be of the liquid or solid tumor type. Liquid tumors include tumors of hematological origin, including, e.g., myelomas (e.g., multiple myeloma), leukemias (e.g., Waldenstrom's syndrome, chronic lymphocytic leukemia, other leukemias), and lymphomas (e.g., B-cell lymphomas, non-Hodgkin's lymphoma). Solid tumors can originate in organs and include cancers of the lungs, brain, breasts, prostate, ovaries, colon, kidneys and liver.

The terms “cancer cell” or “tumor cell” can refer to cells that divide at an abnormal (i.e., increased) rate. Cancer cells include, but are not limited to, carcinomas, such as squamous cell carcinoma, non-small cell carcinoma (e.g., non-small cell lung carcinoma), small cell carcinoma (e.g., small cell lung carcinoma), basal cell carcinoma, sweat gland carcinoma, sebaceous gland carcinoma, adenocarcinoma, papillary carcinoma, papillary adenocarcinoma, cystadenocarcinoma, medullary carcinoma, undifferentiated carcinoma, bronchogenic carcinoma, melanoma, renal cell carcinoma, hepatoma-liver cell carcinoma, bile duct carcinoma, cholangiocarcinoma, papillary carcinoma, transitional cell carcinoma, choriocarcinoma, semonoma, embryonal carcinoma, mammary carcinomas, gastrointestinal carcinoma, colonic carcinomas, bladder carcinoma, prostate carcinoma, and squamous cell carcinoma of the neck and head region; sarcomas, such as fibrosarcoma, myxosarcoma, liposarcoma, chondrosarcoma, osteogenic sarcoma, chordosarcoma, angiosarcoma, endotheliosarcoma, lymphangiosarcoma, synoviosarcoma and mesotheliosarcoma; hematologic cancers, such as myelomas, leukemias (e.g., acute myelogenous leukemia, chronic lymphocytic leukemia, granulocytic leukemia, monocytic leukemia, lymphocytic leukemia), lymphomas (e.g., follicular lymphoma, mantle cell lymphoma, diffuse large B-cell lymphoma, malignant lymphoma, plasmocytoma, reticulum cell sarcoma, or Hodgkin's disease), and tumors of the nervous system including glioma, glioblastoma multiform, meningoma, medulloblastoma, schwannoma and epidymoma.

The term “homology” and “identity” are used synonymously throughout and refer to sequence similarity between two peptides or between two nucleic acid molecules. Homology can be determined by comparing a position in each sequence, which may be aligned for purposes of comparison. When a position in the compared sequence is occupied by the same base or amino acid, then the molecules are homologous or identical at that position. A degree of homology or identity between sequences is a function of the number of matching or homologous positions shared by the sequences.

The term “mutant” refers to any change in the genetic material of an organism, in particular a change (i.e., deletion, substitution, addition, or alteration) in a wild type polynucleotide sequence or any change in a wild type protein. The term “variant” is used interchangeably with “mutant”. Although it is often assumed that a change in the genetic material results in a change of the function of the protein, the terms “mutant” and “variant” refer to a change in the sequence of a wild type protein regardless of whether that change alters the function of the protein (e.g., increases, decreases, imparts a new function), or whether that change has no effect on the function of the protein (e.g., the mutation or variation is silent).

The term “nucleic acid” refers to polynucleotides, such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides.

The phrases “parenteral administration” and “administered parenterally” are art-recognized terms, and include modes of administration other than enteral and topical administration, such as injections, and include, without limitation, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrasternal injection and infusion.

The phrases “systemic administration,” “administered systemically,” “peripheral administration” and “administered peripherally” as used herein mean the administration of a compound, agent or other material other than directly into a specific tissue, organ, or region of the subject being treated (e.g., brain), such that it enters the animal's system and, thus, is subject to metabolism and other like processes, for example, subcutaneous administration.

The terms “patient”, “subject”, “mammalian host,” and the like are used interchangeably herein, and refer to mammals, including human and veterinary subjects.

The terms “peptide(s)”, “protein(s)” and “polypeptide(s)” are used interchangeably herein. As used herein, “polypeptide” refers to any peptide or protein comprising two or more amino acids joined to each other by peptide bonds or modified peptide bonds (i.e., peptide isomers). “Polypeptide(s)” refers to both short chains, commonly referred as peptides, oligopeptides or oligomers, and to longer chains generally referred to as proteins.

The terms “polynucleotide sequence” and “nucleotide sequence” are also used interchangeably herein.

“Recombinant,” as used herein, means that a protein is derived from a prokaryotic or eukaryotic expression system.

The phrase “therapeutically effective amount” or “pharmaceutically effective amount” is an art-recognized term. In certain embodiments, the term refers to an amount of a therapeutic agent that produces some desired effect at a reasonable benefit/risk ratio applicable to any medical treatment. In certain embodiments, the term refers to that amount necessary or sufficient to eliminate, reduce or maintain a target of a particular therapeutic regimen. The effective amount may vary depending on such factors as the disease or condition being treated, the particular targeted constructs being administered, the size of the subject or the severity of the disease or condition. One of ordinary skill in the art may empirically determine the effective amount of a particular compound without necessitating undue experimentation. In certain embodiments, a therapeutically effective amount of a therapeutic agent for in vivo use will likely depend on a number of factors, including: the rate of release of an agent from a polymer matrix, which will depend in part on the chemical and physical characteristics of the polymer; the identity of the agent; the mode and method of administration; and any other materials incorporated in the polymer matrix in addition to the agent.

The term “wild type” refers to the naturally-occurring polynucleotide sequence encoding a protein, or a portion thereof, or protein sequence, or portion thereof, respectively, as it normally exists in vivo.

Throughout the description, where compositions are described as having, including, or comprising, specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

Embodiments described herein relate to near-infrared imaging agents for use in detecting, monitoring, and/or imaging cancer cells and/or cancer cell metastasis, migration, dispersal, and/or invasion in a subject, methods of detecting, monitoring, and/or imaging cancer cells and/or cancer cell metastasis, migration, dispersal, and/or invasion in a subject, methods of determining and/or monitoring the efficacy of a cancer therapeutic and/or cancer therapy administered to a subject in need thereof, methods of determining, monitoring, and/or imaging efficacy of surgical resection of cancer cells in a subject, and/or methods of treating cancer in a subject in need thereof.

Surgical removal of solid tumors is often the first step in cancer treatment. Much effort has been spent recently in developing surgical tools that allow better detection of tumor margins and identification of invasion and metastasis. Recent advances in this area include the development of molecularly-targeted fluorescent imaging agents that aid the surgeon in accurately distinguishing normal from neoplastic tissue in real-time. Of particular importance is the use of Near-Infrared (NIR) fluorophores which provide greater depth of light penetration and are detected at wavelengths where autofluorescence or interference from hemoglobin and other endogenous components is minimal. Glioblastoma (GBM) is among the most challenging tumor types to surgically remove due to its highly invasive and infiltrative characteristics. In GBM, tumor-specific extracellular fragments of PTPμ are generated. PTPμ is a homophilic cell adhesion molecule and a receptor protein tyrosine phosphatase that is normally localized to cell-cell junctions. These PTPμ extracellular fragments remain associated with both the main tumor mass as well as with migratory tumor cells and serve as tumor biomarkers for GBM and other tumor types. Peptides that bind to this PTPμ biomarker were effectively used as recognition and targeting moieties in pre-clinical imaging of tumors with optical imaging, contrast-enhanced magnetic resonance imaging and ultrasound imaging. We found that selected NIR fluorophores conjugated to a PTPμ-derived peptide via a natural or non-natural linkage can be used as tumor-targeted fluorescent imaging agents for fluorescence-guided resection (FGR) with high specificity and favorable kinetics, and show promise for use in the operating room during FGR.

Moreover, it was found that the near-infrared fluorophore that is directly or indirectly links the targeting peptide can be selected such that the near-infrared imaging agent has a structure effective to at least maintain, preserve, or not interfere with binding affinity of the linked targeting peptide to the proteolytically cleaved extracellular fragment and activity of the near-infrared fluorophore. By activity of the near-infrared fluorophore it is meant, for example, the ability of the near-infrared fluorophore to be detected or imaged in vivo, ex vivo, or in vitro by fluorescent imaging so as to provide a signal to background ratio upon fluorescent imaging effective to delineate the cancer cell or another cell in the cancer cell microenvironment from surrounding tissue.

For example, the near-infrared imaging agents described herein were found to clearly demarcate tumor cells in tissue sections and tumor “edge” samples, suggesting that the near-infrared imaging agent can be used as a diagnostic tool for molecular imaging of metastatic, dispersive, migrating, or invading cancers or the tumor margin. Systemic introduction of the near-infrared imaging agent as described herein resulted in rapid and specific labeling of the flank tumors and intracranial tumors within minutes. Labeling occurred primarily within the tumor, however a gradient of agent at the tumor margin was also observed. There is also a signal amplification effect as extracellular fragments accumulate over time.

Accordingly, in some embodiments, a near-infrared imaging agent described herein can include a targeting peptide that specifically binds to and/or complexes with a proteolytically cleaved extracellular fragment of an immunoglobulin (Ig) superfamily cell adhesion molecule that is expressed by a cancer cell or another cell in the cancer cell microenvironment, an optional spacer directly linked to the targeting peptide, and a near-infrared fluorophore that is directly or indirectly linked to the targeting peptide or optional spacer via a natural or non-natural linkage.

The near-infrared imaging agent can be administered locally (e.g., topically) or systemically (e.g., intravenously) to a subject and readily target cancer cells associated with proteolytically cleaved extracellular fragments of the immunoglobulin (Ig) superfamily cell adhesion molecule, such as metastatic, migrating, dispersed, and/or invasive cancer cells. In some embodiments, the near-infrared imaging agent after systemic administration can cross the blood brain barrier to define cancer cell location, distribution, metastases, dispersions, migrations, and/or invasion as well as tumor cell margins in the subject. In other embodiments, the near-infrared imaging agent can be used to image guide phototherapy to inhibit and/or reduce cancer cell survival, proliferation, and migration.

In some embodiments, the near-infrared imaging agents described herein can be used in a method of detecting cancer cells and/or cancer cell metastasis, migration, dispersal, and/or invasion as well as in a method of treating cancer in a subject in need thereof. The methods can include administering to a subject a near-infrared imaging agent that includes a targeting peptide that binds to and/or complexes with the proteolytically cleaved extracellular fragment of the Ig superfamily cell adhesion molecule in the cancer cell or tumor cell microenvironment, an optional spacer directly linked to the targeting peptide. and a near-infrared fluorophore that is directly or indirectly linked to the targeting peptide or optional spacer via a natural or non-natural linkage. The near-infrared imaging agent bound to and/or complexed with the cancer cells can be detected to determine the location and/or distribution of the cancer cells in the subject and/or irradiated for phototherapy and ablation of the cancer cells.

In some embodiments, the Ig superfamily cell adhesion molecule can include an extracellular homophilic binding portion, which can bind in homophilic fashion or engage in homophilic binding in a subject. In one example, the Ig superfamily cell adhesion molecule includes RPTP type IIb cell adhesion molecules. In another example, Ig superfamily cell adhesion molecules can include RPTPs of the PTPμ-like subfamily, such as PTPμ, PTPκ, PTPρ, and PCP-2 (also called PTPλ). PTPμ-like RPTPs include a MAM (Meprin/A5-protein/PTPμ) domain, an Ig domain, and FNIII repeats. PTPμ can have the amino acid sequence of SEQ ID NO: 1, which is identified by Genbank Accession No. AAI51843.1. It will be appreciated that the PTPμ gene can generate splice variants such that the amino acid sequence of PTPμ can differ from SEQ ID NO: 1. In some embodiments, PTPμ can have an amino acid sequence identified by Genbank Accession No. AAH51651.1 and Genbank Accession No. AAH40543.1.

Cancer cells and/or endothelial cells, which support cancer cell survival, that express an Ig superfamily cell adhesion molecule and that can be proteolytically cleaved to produce a detectable extracellular fragment can include, for example, cancer cells and/or other cells in the tumor microenvironment, such as stem cells, endothelial cells, stromal cells and immune cells that promote their survival.

The cancers detected and/or treated by the near-infrared imaging agents described herein can include the following: leukemias, such as but not limited to, acute leukemia, acute lymphocytic leukemia, acute myelocytic leukemias, such as, myeloblastic, promyelocytic, myelomonocytic, monocytic, and erythroleukemia leukemias and myelodysplastic syndrome; chronic leukemias, such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including but not limited to ductal carcinoma, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer such as but not limited to pheochromocytoma and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma: ovarian cancers such as but not limited to, ovarian epithelial carcinoma, borderline tumor, germ cell tumor, and stromal tumor; esophageal cancers such as but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma; gallbladder cancers such as adenocarcinoma; cholangiocarcinomas such as but not limited to papillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, prostatic intraepithelial neoplasia, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell carcinoma, adenocarcinoma, hypemephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J. B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

The near-infrared imaging agent can also be used to detect and/or treat a variety of cancers or other abnormal proliferative diseases, including (but not limited to) the following: carcinoma, including that of the bladder, breast, prostate, rectal, colon, kidney, liver, lung, ovary, pancreas, stomach, cervix, thyroid and skin; including squamous cell carcinoma; hematopoietic tumors of lymphoid lineage, including leukemia, acute lymphocytic leukemia, acute lymphoblastic leukemia, B-cell lymphoma, T-cell lymphoma, Burkitt's lymphoma; hematopoictic tumors of myeloid lineage, including acute and chronic myelogenous leukemias and promyclocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyoscarama, and osteosarcoma; and other tumors, including melanoma, xeroderma pigmentosum, keratoactanthoma, seminoma, thyroid follicular cancer and teratocarcinoma. It is also contemplated that cancers caused by aberrations in apoptosis would also be treated by the methods and compositions of the invention. Such cancers may include but not be limited to follicular lymphomas, carcinomas, hormone dependent tumors of the breast, prostate and ovary, and precancerous lesions such as familial adenomatous polyposis, and myelodysplastic syndromes. In specific embodiments, malignancy or dysproliferative changes (such as metaplasias and dysplasias), or hyperproliferative disorders, are detected, treated, or prevented in the skin, lung, colon, rectum, breast, prostate, bladder, kidney, pancreas, ovary, or uterus. In other specific embodiments, sarcoma, melanoma, or leukemia is detected and/or treated.

In still other embodiments, the cancer cells that are detected and/or treated can include glioma cells, lung cancer cells, breast cancer cells, prostate cancer cells, and melanoma cells, such as invasive, dispersive, motile or metastic cancer cells can include glioma cells, lung cancer cells, breast cancer cells, prostate cancer cells, and melanoma cells. It will be appreciated that other cancer cells and/or endothelial cells, which support cancer cell survival, that express an Ig superfamily cell adhesion molecule and that can be proteolytically cleaved to produce a detectable extracellular fragment can identified or determined by, for example, using immunoassays that detect the Ig superfamily cell adhesion molecule expressed by the cancer cells or endothelial cells.

In some embodiments, the targeting peptide (or targeting polypeptide) can include a polypeptide (or targeting polypeptide) that binds to and/or complexes with the proteolytically cleaved extracellular fragment of the Ig superfamily cell adhesion molecule. The targeting peptide can include, consist essentially of, or consist of about 10 to about 50 amino acids and have an amino acid sequence that is substantially homologous to about 10 to about 50 consecutive amino acids of a homophilic binding portion or domain of the proteleolytically cleaved extracellular fragment of the Ig superfamily cell adhesion molecule. By substantially homologous, it is meant the targeting polypeptide has at least about 80%, about 90%, about 95%, about 96%, about 97%, about 98%, about 99% or about 100% sequence identity with a portion of the amino acid sequence of the binding portion of the proteleolytically cleaved extracellular fragment of the Ig superfamily cell adhesion molecule.

In one example, the homophilic binding portion of the Ig superfamily cell adhesion molecule can include, for example, the Ig domain of the cell adhesion molecule. In another example, where the Ig superfamily cell adhesion molecule is PTPμ, the homophilic binding portion can include the Ig binding domain and the MAM domain.

In another aspect, the targeting peptide can have an amino acid sequence that is substantially homologous to about 10 to about 50 consecutive amino acids of the Ig binding domain and/or MAM domain of PTPμ (e.g., SEQ ID NO: 1) and readily cross the blood brain barrier when systemically administered to a subject. The development of the PTPμ targeting peptides can be based on a large body of structural and functional data. The sites required for PTPμ-mediated homophilic adhesion have been well characterized. In addition, the crystal structure of PTPμ can provide information regarding which regions of each functional domain are likely to be exposed to the outside environment and therefore available for homophilic binding and thus detection by a peptide.

In some embodiments, the proteolytically cleaved extracellular fragment of PTPμ (e.g., SEQ ID NO: 1) can include an amino acid sequence of SEQ ID NO: 2, the Ig and MAM binding region can comprise the amino acid sequence of SEQ ID NO: 3, and the polypeptide can have an amino acid sequence that is substantially homologous to about 10 to about 50 consecutive amino acids of SEQ ID NO: 2 or SEQ ID NO: 3. Examples of polypeptides that can specifically bind SEQ ID NO: 2 or SEQ ID NO: 3 and have an amino acid sequence that is substantially homologous to about 10 to about 50 consecutive amino acids of SEQ ID NO: 2 or SEQ ID NO: 3 are polypeptides that include an amino acid sequence selected from the group consisting of SEQ ID NO: 4, SEQ ID NO: 5 (SBK2), SEQ ID NO: 6, and SEQ ID NO: 7. Polypeptides comprising SEQ ID NO: 4, 5, 6, or 7 can recognize or bind to the MAM, Ig domain, or the FNIII repeats. In particular embodiments, the targeting peptide is a SBK2 polypeptide comprising an amino acid sequence SEQ ID NO: 5.

In other embodiments, a polypeptide that binds to and/or complexes with the proteolytically cleaved extracellular fragment of the Ig superfamily CAM or its receptor that is expressed by a cancer cell or another cell in the cancer cell microenvironment can have an amino acid sequence of SEQ ID NO: 8. SEQ ID NO: 8 is substantially homologous to a portion of SEQ ID NO: 1 or SEQ ID NO: 2 and can specifically bind to SEQ ID NO: 2 or SEQ ID NO: 3.

The targeting peptides can be subject to various changes, substitutions, insertions, and deletions where such changes provide for certain advantages in its use. In this regard, targeting peptides that bind to and/or complex with a proteolytically cleaved extracellular portion of an Ig superfamily cell adhesion molecule can be substantially homologous with, rather than be identical to, the sequence of a recited polypeptide where one or more changes are made and it retains the ability to function as specifically binding to and/or complexing with the proteolytically cleaved extracellular portion of an Ig superfamily cell adhesion molecule.

The targeting peptides can be in any of a variety of forms of polypeptide derivatives, that include amides, conjugates with proteins, cyclized polypeptides, polymerized polypeptides, retro-inverso peptides, analogs, fragments, chemically modified polypeptides, and the like derivatives.

The term “analog” includes any polypeptide having an amino acid residue sequence substantially identical to a sequence specifically shown herein in which one or more residues have been conservatively substituted with a functionally similar residue and that specifically binds to and/or complexes with the proteolytically cleaved extracellular portion of an Ig superfamily CAM as described herein. Examples of conservative substitutions include the substitution of one non-polar (hydrophobic) residue, such as isoleucine, valine, leucine or methionine for another, the substitution of one polar (hydrophilic) residue for another, such as between arginine and lysine, between glutamine and asparagine, between glycine and serine, the substitution of one basic residue such as lysine, arginine or histidine for another, or the substitution of one acidic residue, such as aspartic acid or glutamic acid for another.

The phrase “conservative substitution” also includes the use of a chemically derivatized residue in place of a non-derivatized residue provided that such peptide displays the requisite binding activity.

“Chemical derivative” refers to a subject polypeptide having one or more residues chemically derivatized by reaction of a functional side group. Such derivatized molecules include for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as chemical derivatives are those polypeptides, which contain one or more naturally occurring amino acid derivatives of the twenty standard amino acids. For examples: 4-hydroxyproline may be substituted for proline; 5-hydroxylysine may be substituted for lysine; 3-methylhistidine may be substituted for histidine; homoserine may be substituted for serine; and ornithine may be substituted for lysine. Polypeptides described herein also include any polypeptide having one or more additions and/or deletions or residues relative to the sequence of a polypeptide whose sequence is shown herein, so long as the requisite activity is maintained.

Retro-inverso peptides are linear peptides whose amino acid sequence is reversed and the α-center chirality of the amino acid subunits is inverted as well. These types of peptides are designed by including D-amino acids in the reverse sequence to help maintain side chain topology similar to that of the original L-amino acid peptide and make them more resistant to proteolytic degradation. D-amino acids represent conformational mirror images of natural L-amino acids occurring in natural proteins present in biological systems. Peptides that contain D-amino acids have advantages over peptides that just contain L-amino acids. In general, these types of peptides are less susceptible to proteolytic degradation and have a longer effective time when used as pharmaceuticals. Furthermore, the insertion of D-amino acids in selected sequence regions as sequence blocks containing only D-amino acids or in-between L-amino acids allows the design of peptide based drugs that are bioactive and possess increased bioavailability in addition to being resistant to proteolysis. Furthermore, if properly designed, retro-inverso peptides can have binding characteristics similar to L-peptides.

The term “fragment” refers to any subject polypeptide having an amino acid residue sequence shorter than that of a polypeptide whose amino acid residue sequence is shown herein.

Any polypeptide or compound may also be used in the form of a pharmaceutically acceptable salt. Acids, which are capable of forming salts with the polypeptides, include inorganic acids such as trifluoroacetic acid (TFA) hydrochloric acid (HCl), hydrobromic acid, perchloric acid, nitric acid, thiocyanic acid, sulfuric acid, phosphoric acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid or the like.

Bases capable of forming salts with the polypeptides include inorganic bases such as sodium hydroxide, ammonium hydroxide, potassium hydroxide and the like; and organic bases such as mono-, di- and tri-alkyl and aryl-amines (e.g., triethylamine, diisopropylamine, methylamine, dimethylamine and the like) and optionally substituted ethanolamines (e.g., ethanolamine, diethanolamine and the like).

The targeting peptides can be synthesized by any of the techniques that are known to those skilled in the peptide art, including recombinant DNA techniques. Synthetic chemistry techniques, such as a solid-phase Merrifield-type synthesis, can be used for reasons of purity, antigenic specificity, freedom from undesired side products, ease of production and the like. A summary of the many techniques available can be found in Steward et al., “Solid Phase Peptide Synthesis”, W. H. Freeman Co., San Francisco, 1969; Bodanszky, et al., “Peptide Synthesis”, John Wiley & Sons, Second Edition, 1976; J. Meienhofer, “Hormonal Proteins and Peptides”, Vol. 2, p. 46, Academic Press (New York), 1983; Merrifield, Adv. Enzymol., 32:221-96, 1969; Fields et al., int. J. Peptide Protein Res., 35:161-214, 1990; and U.S. Pat. No. 4,244,946 for solid phase peptide synthesis, and Schroder et al., “The Peptides”, Vol. 1, Academic Press (New York), 1965 for classical solution synthesis, each of which is incorporated herein by reference. Appropriate protective groups usable in such synthesis are described in the above texts and in J. F. W. McOmie, “Protective Groups in Organic Chemistry”, Plenum Press, New York, 1973, which is incorporated herein by reference.

In general, the solid-phase synthesis methods contemplated comprise the sequential addition of one or more amino acid residues or suitably protected amino acid residues to a growing peptide chain. Normally, either the amino or carboxyl group of the first amino acid residue is protected by a suitable, selectively removable protecting group. A different, selectively removable protecting group is utilized for amino acids containing a reactive side group such as lysine.

Using a solid phase synthesis as an example, the protected or derivatized amino acid can be attached to an inert solid support through its unprotected carboxyl or amino group. The protecting group of the amino or carboxyl group can then be selectively removed and the next amino acid in the sequence having the complimentary (amino or carboxyl) group suitably protected is admixed and reacted under conditions suitable for forming the amide linkage with the residue already attached to the solid support. The protecting group of the amino or carboxyl group can then be removed from this newly added amino acid residue, and the next amino acid (suitably protected) is then added, and so forth. After all the desired amino acids have been linked in the proper sequence, any remaining terminal and side group protecting groups (and solid support) can be removed sequentially or concurrently, to afford the final linear polypeptide.

It will be appreciated that the targeting peptide can bind to and/or complex with homophilic binding domains of proteolytically cleaved extracellular fragments of other Ig superfamily cell adhesion molecules, besides PTPs. For example, a similar molecular detection strategy described herein can be used with any other Ig superfamily CAM having a homophilic binding cell surface protein whose ligand binding site is known. A large variety of cell surface proteins, including other phosphatases, are cleaved at the cell surface (Streuli M, Saito H (1992) Expression of the receptor-linked protein tyrosine phosphatase LAR: proteolytic cleavage and shedding of the CAM-like extracellular region. EMBO J 11:897-907; Anders L, Ullrich A (2006) Furin-, ADAM 10-, and gamma-secretase-mediated cleavage of a receptor tyrosine phosphatase and regulation of beta-catenin's transcriptional activity. Mol Cell Biol 26:3917-3934; Haapasalo A, Kovacs D M (2007) Presenilin/gamma-secretase-mediated cleavage regulates association of leukocyte-common antigen-related (LAR) receptor tyrosine phosphatase with beta-catenin. J Biol Chem 282:9063-9072; Chow J P, Noda M (2008) Plasmin-mediated processing of protein tyrosine phosphatase receptor type Z in the mouse brain. Neurosci Lett 442:208-212; Craig S E, Brady-Kalnay S M. Tumor-derived extracellular fragments of receptor protein tyrosine phosphatases (RPTPs) as cancer molecular diagnostic tools. Anticancer Agents Med Chem. 2011 January; 11 (1): 133-40. Review. PubMed PMID: 21235433; PubMed Central PMCID: PMC3337336; Craig S E, Brady-Kalnay S M. Cancer cells cut homophilic cell adhesion molecules and run. Cancer Res. 2011 Jan. 15; 71 (2): 303-9. Epub 2010 Nov. 17. PubMed PMID: 21084269; PubMed Central PMCID: PMC3343737; Phillips-Mason P J, Craig S E, Brady-Kalnay S M. Should I stay or should I go? Shedding of RPTPs in cancer cells switches signals from stabilizing cell-cell adhesion to driving cell migration. Cell Adh Migr. 2011 Jul. 1; 5 (4): 298-305. Epub 2011 Jul. 1. PubMed PMID: 21785275; PubMed Central PMCID: PMC3210297). These proteins represent additional targets for that can be readily used by the skilled artisan for forming therapeutic polypeptides that can be used to treat cancers (Barr A J, Ugochukwu E, Lee W H, King O N, Filippakopoulos P, Alfano I, Savitsky P, Burgess-Brown N A, Muller S, Knapp S (2009) Large-scale structural analysis of the classical human protein tyrosine phosphatome. Cell 136:352-363).

In some embodiments, the targeting peptides described herein can include additional residues that may be added at either terminus of a polypeptide for the purpose of providing a “linker” by which the polypeptides can be conveniently linked and/or affixed to the spacer or near-infrared fluorophore. Typical amino acid residues used for linking are glycine, tyrosine, cysteine, lysine, glutamic and aspartic acid, or the like. In addition, a subject polypeptide can differ by the sequence being modified by terminal-NH₂ acylation, e.g., acetylation, or thioglycolic acid amidation, by terminal-carboxylamidation, e.g., with ammonia, methylamine, and the like terminal modifications. Terminal modifications are useful, as is well known, to reduce susceptibility by proteinase digestion, and therefore serve to prolong half-life of the polypeptides in solutions, particularly biological fluids where proteases may be present. In this regard, polypeptide cyclization is also a useful terminal modification, and is particularly preferred also because of the stable structures formed by cyclization and in view of the biological activities observed for such cyclic peptides as described herein.

The optional spacer can include additional natural and/or non-natural amino acid residues added at either terminus of a targeting peptide (or target peptide with linker peptide). The spacer can include at least three natural or non-natural amino acids and has structure effective to at least maintain or preserve binding affinity of the linked targeting peptide to the proteolytically cleaved extracellular fragment and activity of the near-infrared fluorophore. Typical amino acid residues used for use in the spacer are glycine, serine tyrosine, cysteine, lysine, glutamic and aspartic acid, or the like.

In some embodiments, the spacer is selected in part based on its ability to alter the phobicity (e.g., to cause the agent to become more hydrophilic or hydrophobic) depending on its desired use.

In some embodiments, the spacer can be a flexible peptide that directly or indirectly links the targeting peptide to the near-infrared fluorophore. A flexible peptide or peptidomimetic spacer can be, for example, at least about 3 to about 30 or fewer natural or non-natural amino acids in length. For example, the spacer can have a length of 3, 4, 5, 6, 7, 8, 9, 10, 11, 12, 13, 14, 15, 16, 17, 18, 19, 20, 21, 22, 23, 24, 25, 26, 27, 28, 29, or 30 natural or non-natural amino acids. Where the spacer is a peptide spacer, the peptide spacer may be produced as a single recombinant polypeptide using a conventional molecular biological/recombinant DNA method.

In some embodiments, the peptide spacer includes at least about 50%, at least about 60%, at least about 70%, at least about 80%, or at least about 90% glycine and/or serine residues.

In other embodiments, the peptide spacer includes at least 50%, at least 60%, at least 70%, or at least 80% glycine residues. In some embodiments, the balance of the peptide spacer includes serine residues.

In some embodiments, the peptide spacer is a polyglycine or glycine/serine spacer that consists of purely glycine residues or glycine and serine residues. The small size of glycine residues provides flexibility and allows mobility of the connecting targeting peptide and at least one of detectable moiety, therapeutic agent, or theranostic agent. The incorporation of serine can maintain the stability of the spacer in aqueous solutions by forming hydrogen bonds with water molecules and therefore can reduce unfavorable interactions between the spacer and targeting peptide.

In some embodiments, the peptide spacer includes the amino acid sequence of at least one of (GS)a, (GGS)b, or (GGGS)c, or (GGGGS)d and wherein a, b, c, and d are each independently 2, 3, 4, 5, or 6. For example, the spacer can have an amino acid sequence of

(SEQ ID NO: 9)
GGG,
(SEQ ID NO: 10)
GGGG,
(SEQ ID NO: 11)
GGGGG,
(SEQ ID NO: 12)
GGGGGG,
(SEQ ID NO: 13)
GGGGGGG,
(SEQ ID NO: 14)
GGGGGGGG,
(SEQ ID NO: 15)
GGGGGGGGG,
(SEQ ID NO: 16)
GSGS,
(SEQ ID NO: 17)
GSGSGS,
(SEQ ID NO: 18)
GSGSGSGS,
(SEQ ID NO: 19)
GSGSGSGSGS,
(SEQ ID NO: 20)
GGSGGS,
(SEQ ID NO: 21)
GGSGGSGGS,
(SEQ ID NO: 22)
GGSGGSGGSGGS,
(SEQ ID NO: 23)
GGGSGGGS,
(SEQ ID NO: 24)
GGGSGGGSGGGS,
(SEQ ID NO: 25)
GGGSGGGSGGGSGGGS,
(SEQ ID NO: 26)
GGGGSGGGGS,
or
(SEQ ID NO: 27)
GGGGSGGGGSGGGGS.

In some embodiments, the peptide spacer can be a contiguous portion of the targeting peptide that is coupled to directly to an N terminus or C terminus residue of the targeting peptide with or without a linker peptide.

For example, a polyglycine or glycine/serine space coupled to a SBK2 targeting peptide having SEQ ID NO: 5 can have thee amino acid sequence of

(SEQ ID NO: 28)
GGG.GEGDDFNWEQVNTLTKPTSD,
(SEQ ID NO: 29)
GGGG.GEGDDFNWEQVNTLTKPTSD,
(SEQ ID NO: 30)
GGGGG.GEGDDFNWEQVNTLTKPTSD,
(SEQ ID NO: 31)
GGGGGG.GEGDDFNWEQVNTLTKPTSD,
(SEQ ID NO: 32)
GGGGGGG.GEGDDFNWEQVNTLTKPTSD,
(SEQ ID NO: 33)
GGGGGGGG.GEGDDFNWEQVNTLTKPTSD,
(SEQ ID NO: 34)
GGGGGGGGG.GEGDDFNWEQVNTLTKPTSD,
(SEQ ID NO: 35)
GSGS.GEGDDFNWEQVNTLTKPTSD,
(SEQ ID NO: 36)
GSGSGS.GEGDDFNWEQVNTLTKPTSD,
(SEQ ID NO: 37)
GSGSGSGS.GEGDDFNWEQVNTLTKPTSD,
(SEQ ID NO: 38)
GSGSGSGSGS.GEGDDFNWEQVNTLTKPTSD,
(SEQ ID NO: 39)
GGSGGS.GEGDDFNWEQVNTLTKPTSD,
(SEQ ID NO: 40)
GGSGGSGGS.GEGDDFNWEQVNTLTKPTSD,
(SEQ ID NO: 41)
GGSGGSGGSGGS.GEGDDFNWEQVNTLTKPTSD,
(SEQ ID NO: 42)
GGGSGGGS.GEGDDFNWEQVNTLTKPTSD,
(SEQ ID NO: 43)
GGGSGGGSGGGS.GEGDDFNWEQVNTLTKPTSD,
(SEQ ID NO: 44)
GGGSGGGSGGGSGGGS.GEGDDFNWEQVNTLTKPTSD,
(SEQ ID NO: 45)
GGGGSGGGGS.GEGDDFNWEQVNTLTKPTSD,
or
(SEQ ID NO: 46)
GGGGSGGGGSGGGGS.GEGDDFNWEQVNTLTKPTSD.

It will be appreciated that other spacers can be linked to SBK2 or other targeting peptides described herein at N terminus or C terminus portion of the targeting peptide.

In some embodiments, targeting peptides with a contiguous peptide spacer can be produced as a recombinant polypeptide. For the production of recombinant polypeptides, a variety of host organisms may be used. Examples of hosts include, but are not limited to: bacteria, such as E. coli, yeast cells, insect cells, plant cells and mammalian cells. The skilled artisan will understand how to take into consideration certain criteria in selecting a suitable host for producing the recombinant polypeptide. Factors affecting selection of a host include, for example, post-translational modifications, such as phosphorylation and glycosylation patterns, as well as technical factors, such as the general expected yield and the ease of purification. Host-specific post-translational modifications of the targeting peptide or spacer peptide, which is to be used in vivo, should be carefully considered because certain post-translational modifications are known to be highly immunogenic.

Alternatively, the spacer may be a non-amino or non-peptide linker. For example, the non-peptide linker can be a biocompatible polymer including two or more repeating units linked to each other. Examples of the non-peptide polymer include but are not limited to: polyethylene glycol (PEG), polypropylene glycol (PPG), co-poly (ethylene/propylene) glycol, polyoxyethylene (POE), polyurethane, polyphosphazene, polysaccharides, dextran, polyvinyl alcohol, polyvinylpyrrolidones, polyvinyl ethyl ether, polyacryl amide, polyacrylate, polycyanoacrylates, lipid polymers, chitins, hyaluronic acid, and heparin. Typically, such linkers will have a range of molecular weight of from about 1 kDa to 50 kDa, depending upon a particular linker. For example, a typical PEG has a molecular weight of about 1 to 5 kDa, and polyethylene glycol has a molecular weight of about 5 kDa to 50 kDa, and more preferably about 10 kDa to 40 kDa.

The near-infrared fluorophore that is directly or indirectly linked to the targeting peptide or optional spacer via a natural or non-natural linkage can include an organic small molecule fluorophore that fluoresces upon irradiation in the first near-infrared region (NIR-I, 650-1000 nm) or the second near-infrared region (NIR-II, 1000-1700 nm). The near-infrared fluorophore can be sufficiently or effectively hydrophobic or lipophilic such that when directly or indirectly conjugated to the targeting peptide or optional peptide space with the natural or non-natural linkage, the near-infrared imaging agent has a signal to background ratio (SBR) (or signal to noise ratio (SNR)) upon fluorescent imaging effective to delineate the cancer cell or another cell in the cancer cell microenvironment from surrounding tissue.

It was unexpectedly found that the hydrophobicity or lipophilicity of the organic small molecule near-infrared fluorophore can substantially affect the SBR of the near-infrared imaging agent. More hydrophobic and lipophilic near-infrared fluorophores when directly or indirectly linked to the targeting peptide, which specifically binds to and/or complexes with a proteolytically cleaved extracellular fragment of an immunoglobulin (lg) superfamily cell adhesion molecule, or optional spacer via a natural or non-natural linkage can provide or form a near-infrared imaging agent that can readily delineate cancer cells from surrounding tissue in contrast to more hydrophilic organic small molecule near-infrared fluorophores that when directly or indirectly linked to the targeting peptide or optional spacer via a natural or non-natural linkage could not delineate cancer cells from surrounding tissue.

In some embodiment, the more hydrophobic and lipophilic near-infrared fluorophores, which can provide or form a near-infrared imaging agent with the targeting peptide that can readily delineate cancer cells from surrounding tissue, can include organic small molecule near-infrared fluorophores that have a log P of at least 0, at least about 1, at least about 2, at least about 3, at least about 4, at least about 5, at least about 6 or more, for example, a log P of about 2, about 3, about 4, about 5, about 6, about 7, about 8, about 9, about 10, about 11, about 12, or any range therebetween.

In some embodiments, the near-infrared fluorophore can include a cyanine near-infrared fluorophore having a fluorescence in the first near infrared region or second near infrared region. Cyanine near-infrared fluorophores are broadly defined as two heterocyclic nitrogen atoms that are connected via an electron deficient polymethine bridge. Monomethine cyanines display one methine unit, in this case defined as (═C—), between the heterocyclic structures; this class of compounds displays absorbance within the ultraviolet and visible regions with low fluorescence quantum yield. Elongating the central chromophore length by sets of 2 methylene groups yields tri-, penta-, and heptamethine cyanines. The wavelengths of trimethine cyanines are too low to be effective in NIR imaging in biological systems; however, penta- and heptamethine cyanines have near-infrared absorbance and fluorescence characteristics that are a function of their heterocyclic structure and moieties within the polymethine chain, which can be tuned to offer high quantum yield and molecular brightness.

In some embodiments, the cyanine near-infrared fluorophore that has sufficient hydrophobicity, lipophilicity, and/or log P to provide or form a near-infrared imaging agent with the targeting peptide, which can readily delineate cancer cells from surrounding tissue, can include indocyanine green (ICG) or ICG-Osu.

In some embodiments, the natural or non-natural linker used to directly or indirectly link the near-infrared fluorophore to the targeting peptide can include any natural or chemical linker that is not susceptible to proteolytic cleavage and is a non-contiguous portion of targeting peptide or optional spacer. By a “non-contiguous portion” it is meant that the targeting peptide and spacer are connected via an additional element that is not a part and/or peptide residue of the targeting peptide or spacer that is contiguous in nature and functions as a linker.

In some embodiments, the non-natural linker can be formed using a coupling agent that is attached to or comprises a portion of the near-infrared fluorescent imaging agent. The coupling agent and/or conjugating agent can include, for example, maleimidyl binders, which can be used to bind to thiol groups, isothiocyanate and succinimidyl (e.g., N-hydroxysuccinimidyl (NHS)) binders, which can bind to free amine groups, diazonium which can be used to bind to phenol, and amines, which can be used to bind with free acids such as carboxylate groups using carbodiimide activation. Useful functional groups can be present on the targeting peptide or optional spacer based on the particular amino acids present, and additional groups can be designed. It will be evident to those skilled in the art that a variety of bifunctional or polyfunctional reagents, both homo- and hetero-functional (such as those described in the catalog of the Pierce Chemical Co., Rockford, Ill.), can be employed as a coupling agent. Coupling can be effected, for example, through amino groups, carboxyl groups, sulfhydryl groups or oxidized carbohydrate residues.

Examples of coupling agents and/or conjugating agents are described in Means and Feeney, CHEMICAL MODIFICATION OF PROTEINS, Holden-Day, 1974, pp. 39-43. Among these reagents are, for example, J-succinimidyl 3-(2-pyridyldithio) propionate (SPDP) or N,N′-(1,3-phenylene) bismaleimide (both of which are highly specific for sulfhydryl groups and form irreversible linkages); N,N′-ethylene-bis-(iodoacetamide) or other such reagent having 6 to 11 carbon methylene bridges (which relatively specific for sulfhydryl groups); and 1,5-difluoro-2,4-dinitrobenzene (which forms irreversible linkages with amino and tyrosine groups). Other coupling agents or conjugating can include: p,p′-difluoro-m,m′-dinitrodiphenylsulfone (which forms irreversible linkages with amino and phenolic groups); dimethyl adipimidate (which is specific for amino groups); phenol-1,4-disulfonylchloride (which reacts principally with amino groups); hexamethylenediisocyanate or diisothiocyanate, or azophenyl-p-diisocyanate (which reacts principally with amino groups); glutaraldehyde (which reacts with several different side chains) and disdiazobenzidine (which reacts primarily with tyrosine and histidine).

The coupling agent may be homobifunctional, i.e., having two functional groups that undergo the same reaction. An example of a homobifunctional cross-linking reagent is bismaleimidohexane (“BMH”). BMH contains two maleimide functional groups, which react specifically with sulfhydryl-containing compounds under mild conditions (pH 6.5-7.7). The two maleimide groups are connected by a hydrocarbon chain. Therefore, BMH is useful for irreversible linking of polypeptides that contain cysteine residues.

Coupling agents may also be heterobifunctional. Heterobifunctional coupling or conjugating agents have two different functional groups, for example an amine-reactive group and a thiol-reactive group, that will cross-link two proteins having free amines and thiols, respectively. Examples of heterobifunctional cross-linking agents are succinimidyl 4-(N-maleimidomethyl)cyclohexane-1-carboxylate (“SMCC”), m-maleimidobenzoyl-N-hydroxysuccinimide ester (“MBS”), and succinimide 4-(p-maleimidophenyl) butyrate (“SMPB”), an extended chain analog of MBS. The succinimidyl group of these cross-linkers reacts with a primary amine, and the thiol-reactive maleimide forms a covalent bond with the thiol of a cysteine residue.

The coupling agents can yield a conjugate of the targeting peptide or optional spacer and the near-infrared fluorophore that is essentially non-cleavable under cellular conditions. Numerous coupling agents, including the ones discussed above, are commercially available. Detailed instructions for their use are readily available from the commercial suppliers. A general reference on protein cross-linking and conjugate preparation is: Wong, CHEMISTRY OF PROTEIN CONJUGATION AND CROSS-LINKING, CRC Press (1991).

In some embodiments, the near infrared imaging can have the formula:

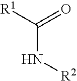

or a pharmaceutically acceptable salt thereof;
wherein R¹ is the near-infrared fluorophore described herein; and
R² includes a targeting peptide that specifically binds to and/or complexes with a proteolytically cleaved extracellular fragment of an immunoglobulin (Ig) superfamily cell adhesion molecule that is expressed by a cancer cell or another cell in the cancer cell microenvironment and an optional spacer directly linked to the targeting peptide.

In other embodiments, the near-infrared imaging agent can include a compound of formula:

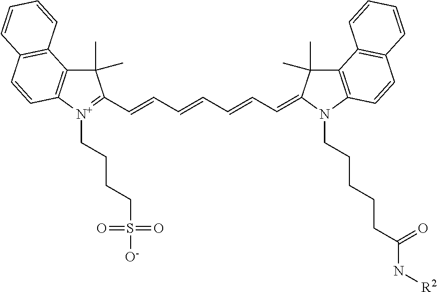

or a pharmaceutically acceptable salt thereof;
wherein R² includes a targeting peptide that specifically binds to and/or complexes with a proteolytically cleaved extracellular fragment of an immunoglobulin (Ig) superfamily cell adhesion molecule that is expressed by a cancer cell or another cell in the cancer cell microenvironment and an optional spacer directly linked to the targeting peptide.

In some embodiment, R² consists of the targeting peptide.

In other embodiments, R² consists of the targeting peptide linked to a spacer and wherein the spacer separates the amide from the targeting peptide.

The near-infrared imaging agents described herein can be administered to the subject by, for example, systemic, topical, and/or parenteral methods of administration. These methods include, e.g., injection, infusion, deposition, implantation, or topical administration, or any other method of administration where access to the tissue by the near-infrared imaging agent is desired. In one example, administration of the near-infrared imaging agent can be by intravenous injection of the near-infrared imaging agent in the subject. Single or multiple administrations of the probe can be given. “Administered”, as used herein, means provision or delivery of the near-infrared imaging agent in an amount(s) and for a period of time(s) effective to label cancer cells in the subject.

Near-infrared imaging agents described herein can be administered to a subject in a detectable quantity of a pharmaceutical composition containing the near-infrared imaging agent or a pharmaceutically acceptable water-soluble salt thereof, to a patient.

Formulation of the near-infrared imaging agent to be administered will vary according to the route of administration selected (e.g., solution, emulsion, capsule, and the like). Suitable pharmaceutically acceptable carriers may contain inert ingredients which do not unduly inhibit the biological activity of the near-infrared imaging agents. The pharmaceutically acceptable carriers should be biocompatible, e.g., non-toxic, non-inflammatory, non-immunogenic and devoid of other undesired reactions upon the administration to a subject. Standard pharmaceutical formulation techniques can be employed, such as those described in Remington's Pharmaceutical Sciences, ibid. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like.

The preparation of a pharmacological composition that contains active ingredients dissolved or dispersed therein is well understood in the art. Typically, such compositions are prepared as injectables either as liquid solutions or suspensions, however, solid forms suitable for solution, or suspensions, in liquid prior to use can also be prepared. Formulation will vary according to the route of administration selected (e.g., solution, emulsion, capsule).

A “detectable quantity” means that the amount of the near-infrared imaging agent that is administered is sufficient to enable detection of binding of the near-infrared imaging agent to the cancer cells. An “imaging effective quantity” means that the amount of the near-infrared imaging agent that is administered is sufficient to enable fluorescent imaging of binding of the near-infrared imaging agent to the cancer cells.

The near-infrared imaging agent administered to a subject can be used in a method to detect and/or determine the presence, location, and/or distribution of cancer cells, i.e., cancer cells associated with proteolytically cleaved extracellular fragments of Ig superfamily cell adhesion molecules, in an organ or body area of a patient, e.g., at least one region of interest (ROI) of the subject. The ROI can include a particular area or portion of the subject and, in some instances, two or more areas or portions throughout the entire subject. The ROI can include regions to be imaged for both diagnostic and therapeutic purposes. The ROI is typically internal; however, it will be appreciated that the ROI may additionally or alternatively be external.

The presence, location, and/or distribution of the near-infrared imaging agent in the animal's tissue, e.g., brain tissue, can be visualized with a near-infrared fluorescence (NIRF) scanner. In one example, the NIRF scanner may be handheld. In another example, the NIRF scanner may be miniaturized and embedded in an apparatus (e.g., micro-machines, scalpel, neurosurgical cell removal device).

“Distribution” as used herein is the spatial property of being scattered about over an area or volume. In this case, “the distribution of cancer cells” is the spatial property of cancer cells being scattered about over an area or volume included in the animal's tissue, e.g., brain tissue. The distribution of the near-infrared imaging agent may then be correlated with the presence or absence of cancer cells in the tissue. A distribution may be dispositive for the presence or absence of a cancer cells or may be combined with other factors and symptoms by one skilled in the art to positively detect the presence or absence of migrating or dispersing cancer cells, cancer metastases or define a tumor margin in the subject. It will be appreciated that the imaging modality may be used to generate a baseline image prior to administration of the near-infrared imaging agent. In this case, the baseline and post-administration images can be compared to ascertain the presence, absence, and/or extent of a particular disease or condition.

In one aspect, the near-infrared imaging agent may be administered to a subject to assess the distribution of cancer cells in a subject and correlate the distribution to a specific location. Surgeons routinely use intra-operative fluorescent imaging in surgical resections. This allows them to specifically identify and sample tissue from distinct regions of the tumor such as the tumor edge or tumor center. Frequently, they also sample regions of brain on the tumor margin that are outside the tumor edge that appear to be grossly normal but are infiltrated by dispersing tumor cells upon histological examination. For example, in glioma (brain tumor) surgery, the near-infrared imaging agent can be given intravenously prior to pre-surgical localization imaging. The agents can be imaged using near-infrared fluorescent imaging that localizes with the glioma.

Agents described herein that include a near-infrared imaging agent and specifically bind to and/or complex with proteolytically cleaved Ig superfamily cell adhesion molecules (e.g., PTPμ) associated with cells can be used in intra-operative imaging (IOI) techniques to guide surgical resection and eliminate the “educated guess” of the location of the tumor margin by the surgeon. Previous studies have determined that more extensive surgical resection improves patient survival. Stummer W, Novotny A. Stepp H, Goetz C, Bise K, Reulen H J (2000) Fluorescence-guided resection of glioblastoma multiforme by using 5-aminolevulinic acid-induced porphyrins: a prospective study in 52 consecutive patients. J Neurosurg 93:1003-1013. Fluorescence-guided resection of glioblastoma multiforme by using 5-aminolevulinic acid-induced porphyrins: a prospective study in 52 consecutive patients. Stummer W, Novotny A, Stepp H, Goetz. C, Bise K, Reulen H J (2000) Fluorescence-guided resection of glioblastoma multiforme by using 5-aminolevulinic acid-induced porphyrins: a prospective study in 52 consecutive patients. J Neurosurg 93:1003-1013. Thus, near-infrared imaging agent that function as diagnostic molecular imaging agents have the potential to increase patient survival rates.

In some embodiments, the near-infrared imaging agent upon administration to the subject can target and detect and/or determine the presence, location, and/or distribution of cancer cells, i.e., cancer cells associated with proteolytically cleaved extracellular fragments of Ig superfamily cell adhesion molecules, in an organ or body area of a patient. In one example, the near-infrared imaging agent can be combined with intraoperative imaging (IOI) to identify malignant cells that have infiltrated and/or are beginning to infiltrate at a tumor brain margin. The method can be performed in real-time during brain or other surgery. The method can include local or systemic application of the near-infrared imaging agent described herein. A fluorescent imaging modality can then be used to detect and subsequently gather image data. The resultant image data may be used to determine, at least in part, a surgical and/or radiological treatment. Alternatively, this image data may be used to control, at least in part, an automated surgical device (e.g., laser, scalpel, micromachine) or to aid in manual guidance of surgery. Further, the image data may be used to plan and/or control the delivery of a therapeutic agent (e.g., by a micro-electronic machine or micro-machine).

In one example, a near-infrared imaging agent can be topically applied as needed during surgery to interactively guide a surgeon and/or surgical instrument to remaining abnormal cells. The near-infrared imaging agent may be applied locally in low concentration, making it unlikely that pharmacologically relevant concentrations are reached. In one example, excess material may be removed (e.g., washed off) after a period of time (e.g., incubation period).

Another embodiment described herein relates to a method of monitoring the efficacy of a cancer therapeutic or cancer therapy administered to a subject. The methods and agents described herein can be used to monitor and/or compare the invasion, migration, dispersal, and metastases of a cancer in a subject prior to administration of a cancer therapeutic or cancer therapy, during administration, or post therapeutic regimen.

A “cancer therapeutic” or “cancer therapy”, as used herein, can include any agent or treatment regimen that is capable of negatively affecting cancer in an animal, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of an animal with cancer. Cancer therapeutics can include one or more therapies such as, but not limited to, chemotherapies, radiation therapies, hormonal therapies, and/or biological therapies/immunotherapies. A reduction, for example, in cancer volume, growth, migration, and/or dispersal in a subject may be indicative of the efficacy of a given therapy. This can provide a direct clinical efficacy endpoint measure of a cancer therapeutic. Therefore, in another aspect, a method of monitoring the efficacy of a cancer therapeutic is provided. More specifically, embodiments of the application provide for a method of monitoring the efficacy of a cancer therapy.

The method of monitoring the efficacy of a cancer therapeutic can include the steps of administering in vivo to the animal a near-infrared imaging agent as described herein, then visualizing a distribution of the near-infrared imaging agent in the animal (e.g., with an in vivo imaging modality as described herein), and then correlating the distribution of the near-infrared imaging agent with the efficacy of the cancer therapeutic. It is contemplated that the administering step can occur before, during, and after the course of a therapeutic regimen in order to determine the efficacy of a chosen therapeutic regimen. One way to assess the efficacy of the cancer therapeutic is to compare the distribution of a near-infrared imaging agent pre and post cancer therapy.

In some embodiments, the near-infrared imaging agent bound to and/or complexed with the proteolytically cleaved extracellular fragment of the Ig superfamily cell adhesion molecule is detected in the subject to detect and/or provide the location and/or distribution of the cancer cells in the subject. The location and/or distribution of the cancer cells in the subject can then be compared to a control to determine the efficacy of the cancer therapeutic and/or cancer therapy. The control can be the location and/or distribution of the cancer cells in the subject prior to the administration of the cancer therapeutic and/or cancer therapy. The location and/or distribution of the cancer cells in the subject prior to the administration of the cancer therapeutic and/or cancer therapy can be determined by administering the near-infrared imaging agent to the subject and detecting the agent bound to and/or complexed with cancer cells in the subject prior to administration of the cancer therapeutic and/or cancer therapy.

In certain embodiments, the methods and agents described herein can be used to measure the efficacy of a therapeutic administered to a subject for treating a metastatic, invasive, or dispersed cancer. In this embodiment, the agent can be administered to the subject prior to, during, or post administration of the therapeutic regimen and the distribution of cancer cells can be imaged to determine the efficacy of the therapeutic regimen. In one example, the therapeutic regimen can include a surgical resection of the metastatic cancer and the near-infrared imaging agent can be used to define the distribution of the metastatic cancer pre-operative and post-operative to determine the efficacy of the surgical resection. Optionally, the methods and near-infrared imaging agents can be used in an intra-operative surgical procedure as describe above, such as a surgical tumor resection, to more readily define and/or image the cancer cell mass or volume during the surgery.

In other embodiments, the near-infrared imaging agent can used in a method of treating cancer or tumors (e.g., brain cancer or tumors). In one embodiment, the near-infrared imaging agent can be used in imaging-mediated phototherapy to ablate cancer cells or another cell in the cancer cell microenvironment.

Image-mediated phototherapy can include imaging-guided photothermal therapy (PTT) and imaging-guided photodynamic therapy (PDT). In PTT, the near-infrared fluorophore of the near-infrared imaging agent bound to the cancer cell or another cell in the cancer cell microenvironment can be irradiated with a wavelength of light effective to convert light energy into heat and ablate the cancer. Advantageously, the as produced heat can potentially cause thermal expansion of the cancer tissue to generate photoacoustic imaging (PAI) signal. Alternatively, the near-infrared fluorophore of the near-infrared imaging agent bound to the cancer cell or another cell in the cancer cell microenvironment can be irradiated with wavelength of light effective produce singlet oxygen (O₂) or other reactive oxygen species (ROS) under laser irradiation to induce apoptosis or necrosis of cancer cells, which can be applied for imaging-guided photodynamic therapy (PDT) or further to achieve synergistic PDT/PTT. Only the cells that are exposed simultaneously to the near-infrared imaging agent and light are destroyed while surrounding healthy, non-targeted and nonirradiated cells are spared from photodamage. Furthermore, the fluorescence of the near-infrared imaging agent enables simultaneous diagnostic optical imaging that can be used to guide the cancer treatment.

Methods for conducting PTT and/or PDT are known in the art. See for example Thierry Patrice. Photodynamic Therapy; Royal Society of Chemistry, 2004. A pharmaceutical composition including near-infrared agent describe herein can be applied to an organ or tissue as a step in PTT and/or PDT. In certain embodiments, the composition is applied to an epithelial, mesothelial, synovial, fascial, or serosal surface, including, but not limited to, the eye, esophagus, mucous membrane, bladder, joint, tendon, ligament, bursa, gastrointestinal, genitourinary, pleural, pericardial, pulmonary, or uroepithelial surfaces.

A near-infrared imaging agent for PTT and/or PDT can also be administered to a subject with cancer by systemic administration, such as intravenous administration. Upon administration, the near-infrared imaging agent described herein can localize to and/or accumulate at the site of the targeted tumor or cancer. In some embodiments, specific binding and/or complexing with a proteolytically cleaved extracellular fragment of an immunoglobulin (Ig) superfamily cell adhesion molecule that is expressed by a cancer cell or another cell in the cancer cell microenvironment allows the agent to be bound to, complexed with and/or taken up by the targeted cells. This binding and/or uptake is specific to the targeted cells, which allows selective targeting of the cancer cells and/or cells in the cancer cell microenvironment in the subject by the targeted agents.

Following administration and localization of the near-infrared imaging agent to the targeted cancer cells, the targeted cancer cells can be exposed to therapeutic amount of light that causes cancer cell ablation, damage and/or suppression of cancer cell growth. The light, which is capable of activating the near-infrared imaging agent for PTT and/or PDT agent can be delivered to the targeted cancer cells using, using for example, semiconductor laser, dye laser, optical parametric oscillator or the like. It will be appreciated that any source light can be used as long as the light excites the near-infrared imaging agent.

The near-infrared imaging agent described herein can be administered to a subject by any conventional method of drug administration, for example, orally in capsules, suspensions or tablets or by parenteral administration. Parenteral administration can include, for example, intramuscular, intravenous, intraventricular, intraarterial, intrathecal, subcutaneous, or intraperitoneal administration. The disclosed compounds can also be administered orally (e.g., in capsules, suspensions, tablets or dietary), nasally (e.g., solution, suspension), transdermally, intradermally, topically (e.g., cream, ointment), inhalation (e.g., intrabronchial, intranasal, oral inhalation or intranasal drops) transmucosally or rectally. Delivery can also be by injection into the brain or body cavity of a patient or by use of a timed release or sustained release matrix delivery systems, or by onsite delivery using micelles, gels and liposomes. Nebulizing devices, powder inhalers, and aerosolized solutions may also be used to administer such preparations to the respiratory tract. Delivery can be in vivo, or ex vivo. Administration can be local or systemic as indicated. More than one route can be used concurrently, if desired. The preferred mode of administration can vary depending upon the particular disclosed compound chosen. In specific embodiments, oral, parenteral, or systemic administration are preferred modes of administration for treatment.

The near-infrared imaging agent described herein can be administered alone as a monotherapy, or in conjunction with or in combination with one or more additional therapeutic agents. For example, the near-infrared imaging agent described herein can be administered to the subject prior to, during, or post administration of an additional therapeutic agent and the distribution of metastatic cells can be targeted with the therapeutic agent. The near-infrared imaging agent can be administered to the animal as part of a pharmaceutical composition comprising the near-infrared imaging agent and a pharmaceutically acceptable carrier or excipient and, optionally, one or more additional therapeutic agents. The near-infrared imaging agent described herein and additional therapeutic agent can be components of separate pharmaceutical compositions, which can be mixed together prior to administration or administered separately. The near-infrared imaging agent described herein, for example, can be administered in a composition containing the additional therapeutic agent, and thereby, administered contemporaneously with the agent. Alternatively, the near-infrared imaging agent and therapeutic agent described herein can be administered contemporaneously, without mixing (e.g., by delivery of the agent on the intravenous line by which the therapeutic agent is also administered, or vice versa). In another embodiment, the near-infrared imaging agent described herein can be administered separately (e.g., not admixed), but within a short time frame (e.g., within 24 hours) of administration of the therapeutic agent.

The methods described herein contemplate single as well as multiple administrations, given either simultaneously or over an extended period of time. The near-infrared imaging agent described herein (or composition containing the agent) can be administered at regular intervals, depending on the nature and extent of the inflammatory disorder's effects, and on an ongoing basis. Administration at a “regular interval,” as used herein, indicates that the therapeutically effective amount is administered periodically (as distinguished from a one-time dose). In one embodiment, the near-infrared imaging agent is administered periodically, e.g., at a regular interval (e.g., bimonthly, monthly, biweekly, weekly, twice weekly, daily, twice a day or three times or more often a day).

The administration interval for a single individual can be fixed, or can be varied over time, depending on the needs of the individual. For example, in times of physical illness or stress, or if disease symptoms worsen, the interval between doses can be decreased.

For example, the administration of the near-infrared imaging agent and/or the additional therapeutic agent can take place at least once on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least once on week 1, 2, 3, 4, 5, 6, 7, 8, 9, 10, 11, 12, 13, 14, 15, 16, 17, 18, 19 or 20, or any combination thereof, using single or divided doses of every 60, 48, 36, 24, 12, 8, 6, 4, or 2 hours, or any combination thereof. Administration can take place at any time of day, for example, in the morning, the afternoon or evening. For instance, the administration can take place in the morning, e.g., between 6:00 a.m. and 12:00 noon; in the afternoon, e.g., after noon and before 6:00 p.m.; or in the evening, e.g., between 6:01 p.m. and midnight.

The near-infrared imaging agent and therapeutic agent described herein and/or additional therapeutic agent can be administered in a dosage of, for example, 0.1 to 100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day. Dosage forms (composition) suitable for internal administration generally contain from about 0.1 milligram to about 500 milligrams of active ingredient per unit. In these pharmaceutical compositions the active ingredient will ordinarily be present in an amount of about 0.5-95% by weight based on the total weight of the composition.

The amount of disclosed near-infrared imaging agent, and therapeutic agent described herein and/or additional therapeutic agent administered to the subject can depend on the characteristics of the subject, such as general health, age, sex, body weight and tolerance to drugs as well as the degree, severity and type of rejection. The skilled artisan will be able to determine appropriate dosages depending on these and other factors using standard clinical techniques.

In addition, in vitro or in vivo assays can be employed to identify desired dosage ranges. The dose to be employed can also depend on the route of administration, the seriousness of the disease, and the subject's circumstances. Effective doses may be extrapolated from dose-response curves derived from in vitro or animal model test systems. The amount of the near-infrared imaging agent described herein can also depend on the disease state or condition being treated along with the clinical factors and the route of administration of the near-infrared imaging agent.

The disclosed near-infrared imaging agent described herein can be administered to the subject in conjunction with an acceptable pharmaceutical carrier or diluent as part of a pharmaceutical composition for therapy. Formulation of the near-infrared imaging agent to be administered will vary according to the route of administration selected (e.g., solution, emulsion, capsule, and the like). Suitable pharmaceutically acceptable carriers may contain inert ingredients which do not unduly inhibit the biological activity of the compounds. The pharmaceutically acceptable carriers should be biocompatible, e.g., non-toxic, non-inflammatory, non-immunogenic and devoid of other undesired reactions upon the administration to a subject. Standard pharmaceutical formulation techniques can be employed, such as those described in Remington's Pharmaceutical Sciences, ibid. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Methods for encapsulating compositions (such as in a coating of hard gelatin or cyclodextran) are known in the art (Baker, et al., “Controlled Release of Biological Active Agents”, John Wiley and Sons, 1986).

The preparation of a pharmacological composition that contains active ingredients dissolved or dispersed therein is well understood in the art. Typically, such compositions are prepared as injectables either as liquid solutions or suspensions, however, solid forms suitable for solution, or suspensions, in liquid prior to use can also be prepared. Formulation will vary according to the route of administration selected (e.g., solution, emulsion, capsule).

A pharmaceutically acceptable carrier for a pharmaceutical composition can also include delivery systems known to the art for entraining or encapsulating drugs, such as anticancer drugs. In some embodiments, the disclosed compounds can be employed with such delivery systems including, for example, liposomes, nanoparticles, nanospheres, nanodiscs, dendrimers, and the like. See, for example Farokhzad, O. C., Jon, S., Khademhosseini, A., Tran, T. N., Lavan, D. A., and Langer, R. (2004). “Nanoparticle-aptamer bioconjugates: a new approach for targeting prostate cancer cells.” Cancer Res., 64, 7668-72; Dass, C. R. (2002). “Vehicles for oligonucleotide delivery to tumours.” J. Pharm. Pharmacol., 54, 3-27; Lysik, M. A., and Wu-Pong, S. (2003). “Innovations in oligonucleotide drug delivery.” J. Pharm. Sci., 92, 1559-73; Shoji, Y., and Nakashima, H. (2004). “Current status of delivery systems to improve target efficacy of oligonucleotides.” Curr. Pharm. Des., 10, 785-96; Allen, T. M., and Cullis, P. R. (2004). “Drug delivery systems: entering the mainstream.” Science, 303, 1818-22. The entire teachings of each reference cited in this paragraph are incorporated herein by reference.

The following example is for the purpose of illustration only and are not intended to limit the scope of the claims, which are appended hereto.

### Example

Recent studies focus on molecular imaging agents that incorporate cancer-specific targeting moieties to allow more precise localization of the fluorescent label to the tumor in vivo. Ideally, these agents will more clearly distinguish tumor margins and aid in identifying invasive or metastatic cells. The PTPμ biomarker, derived from full-length Receptor Protein Tyrosine Phosphatase mu (PTPμ), is found at both tumor edges and in association with dispersing cells in GBM. In normal cells, PTPμ is a homophilic cell adhesion molecule that plays a role in contact inhibition. In GBM and other cancers, PTPμ-mediated cell-cell adhesion is altered through aberrant protease activity which cleaves the extracellular domain of PTPμ into fragments within the tumor microenvironment. Peptides targeting the homophilic binding region within the PTPμ biomarker successfully bind GBM both in vitro and in vivo using multiple imaging modalities. These “SBK” peptides have demonstrated labeling of the main tumor mass as well as migratory and invasive glioma cells using a unique cryoimaging technique following conjugation to the Cy5 fluorophore.

Four PTPμ-derived peptides were utilized as tumor imaging agents. One of those peptides, SBK2, was selected as the targeting moiety for the agents described here based on previous studies. Accurately predicting how a molecular imaging agent will behave in vivo is challenging. In addition to selection of the targeting moiety, both the fluorophore and a spacer or linker greatly influence biodistribution. We tested the insertion of a six amino acid linker GGSGGS (SEQ ID NO: 20) between the targeting peptide and fluorophore. This linker was designed to provide flexibility between the peptide and fluorophore. Three NIR fluorophores were selected to explore the effect of fluorophore hydrophobicity/hydrophilicity on in vivo behavior: ICG, IRDye® 800CW, and Tide Fluor™ 8WS. ICG is a more hydrophobic molecule and has been safely used in the clinic for decades. In contrast, both IRDye® 800CW, and Tide Fluor™ 8WS are hydrophilic dyes according to their manufacturers. As controls, Scrambled peptide versions of all agents were synthesized and tested in mice to verify specificity of the SBK2-containing agents for the PTPμ biomarker in vivo.

### Materials and Methods

### Materials

Fmoc-protected amino acids were purchased from either Aapptec, Louisville, KY, USA or Chem-Impex, Wood Dale, IL, USA. Other reagents obtained from Chem-Impex included 2-chlorotrityl chloride resin, and (0-(6-chlorobenzotriazol-1-yl)-N,N,N′,N′-tetramethyluronium hexafluorophosphate (HCTU). The solvents N,N-dimethylformamide (DMF), dichloromethane (DCM), diethyl ether, HPLC grade water, and HPLC grade acetonitrile were purchased from Fisher Scientific (Pittsburgh, PA, USA). Anhydrous N,N-diisopropylethyl amine (DIPEA), trifluoroacetic acid (TFA), 2,2′-(ethylenedioxy) diethanethiol (DODT), triisopropylsilane (TIS), 4-methyl piperidine, dimethyl sulfoxide (DMSO), and ammonium bicarbonate were obtained from Sigma-Aldrich (St. Louis, MO, USA). Phosphate Buffered Saline (PBS), pH 7.4, from Life Technologies Corp., Grand Island, NY, USA, or 0.9% sodium chloride (saline) from ICU Medical, Lafe Forest, IL, USA, were used to prepare and administer the imaging agents. The fluorophore IRDye® 800CW maleimide was from LI-COR Biosciences, Lincoln, NE, USA. The fluorophores Tide Fluor™ 8WS, indocyanine green-OSu (ICG-OSu) and ICG acid were purchased from AAT Bioquest, Pleasanton, CA, USA.

### Synthesis of Near-Infrared Imaging Agents

The PTPμ-targeted peptide SBK2 and Scrambled control peptide (“Scram”) have been described previously. For some agents, six additional amino acids (GGSGGS) were incorporated at the N-terminus. For brevity, these additional amino acids are referred to as “CLE” in the agent names. Peptides were conjugated on-resin to ICG-OSu. A detailed description of the method used in-house is provided in this example. Some ICG-labeled peptides were made by PolyPeptide Group, San Diego, CA. USA in the same manner.

The concentrations of stocks were determined using the A780 nm of multiple dilutions made into DMSO and the extinction coefficient (€) of 230,000 M⁻¹ cm⁻¹ listed on the manufacturer's website. Multiple dilutions were used to ensure measurements were made in the linear range.

The hydrophilic nature and pH sensitivity of the IRDye® 800CW precluded on-resin coupling in the organic solvents and TFA-based cleavage cocktail used for ICG-labeled molecules. IRDye® 800CW maleimide was conjugated in aqueous solution following the manufacturer's recommended protocol to cysteine residues at the N-termini of the peptides and obtained at >95% purity from PolyPeptide Group. Additional LC-MS/MS analyses using an Orbitrap Elite LC-MS, purchased via an NIH shared instrument grant, 1S10RR031537-01, were performed on these agents by the Lerner Research Institute Proteomics Core at the Cleveland Clinic.

The Tide Fluor™ 8WS-conjugated peptides “SBK2-TF8WS” and “Scram-TF8WS” were made by Bachem Americas, Inc., Torrance CA, USA by addition of the acid form of the Tide Fluor™ 8WS fluorophore to the N-termini of the SBK2 and Scrambled peptides as the final step of synthesis following activation by the solid phase peptide synthesis coupling agents. The agents were obtained at >95% purity.

### Detailed Method for Synthesis of ICG-Labeled SBK2/SBK2-CLE and Scrambled/Scrambled-CLE Peptides

For ICG-labeling of peptides, dry peptidyl resins lacking an N-terminal Fmoc group were weighed to obtain the desired amount of crude peptide, swelled in DMF for at least two hours, and then incubated in 100 mM triethylamine in anhydrous DMF for 10 minutes. Excess solvent was removed from the swelled resin, a 1.5-2-fold molar excess relative to peptide of ICG-OSu, 20 mM in DMSO, was added along with a 10-fold molar excess of triethylamine, and the resin-dye mixture was placed on a rotator, protected from light and allowed to react overnight at room temperature. The peptidyl resin-dye mixture was transferred to a fritted syringe (Torviq, Tucson, AZ, USA), and washed extensively in DMF to remove any unreacted fluorophore, equilibrated in DCM and incubated for 1 h at room temperature in a cleavage cocktail of 92.5% TFA/2.5% TIS/2.5% DODT/2.5% water. After 1 h, the cleaved peptide was precipitated in ice-cold diethyl ether, washed thoroughly with additional diethyl ether, and then allowed to dry. ICG-conjugated peptides were assessed by reverse phase HPLC using an analytical C18 column (Eclipse XDB-C18, 5 μm, 4.6×150 mm, Agilent, Santa Clara, CA, USA) with water/0.1% TFA (solvent A) and acetonitrile/0.1% (solvent B). HPLC runs were monitored at both 220 nm and 780 nm with an SPD-M20A Diode Array Detector (Shimadzu Scientific Instruments, Columbia, MD, USA). ICG-conjugated peptides were purified using a preparative C-18 column (ZORBAX 300SB-C18 PrepHT, 21.2×250 mm, 7 μm column, Agilent, Santa Clara, CA, USA) and the same solvents to >95% purity.

### Mice and Experimental Tumor Models

All applicable institutional and/or NIH guidelines for the care and use of animals were followed. The Case Western Reserve University Institutional Animal Care and Use Committee reviewed and approved the animal protocols. Outbred homozygous female nude mice (Fox1^{nu}/Fox1^{nu}) were purchased from Jackson Laboratory, Bar Harbor, ME, USA. Details of the human glioblastoma LN-229 cell line have been described previously. LN-229 cells express the PTPμ biomarker and have been used in previous studies for evaluating PTPμ-targeted agents using different imaging modalities. Flank tumors were started in the right flank of mice at approximately 7 weeks of age by injecting a mixture of 2×10⁶ cells and Matrigel® Matrix (Corning®, Corning Inc., Corning, NY, USA). Experiments were performed in mice 4 to 8 weeks after implanting the tumors.

### Fluorescent Imaging Experiments

The IVIS Spectrum In vivo Imaging System and Living Image analysis software, (Perkin Elmer, Hopkinton, MA, USA) were used for acquisition and analysis of mouse images. Prior to the experiment, mice were weighed, and baseline images acquired using the appropriate filter sets and the autoexposure setting. Three filter sets for excitation/emission were used: 745 nm/800 nm, 745 nm/820 nm, 745 nm/840 nm. The filter set showing highest fluorescence intensity for a fluorophore was used throughout for that dye. For ICG, 745 nm/820 nm was used and for IRDye® 800CW and Tide Fluor™ 8WS, 745 nm/800 nm was used. Agent stocks were diluted in saline or PBS to ˜100 μM and the volume needed for the desired dose of 300 nmol/kg or 400 nmol/kg calculated. Doses were loaded into polyurethane tubing and connected to a 1 mL syringe as previously described. Mice were anesthetized in an induction chamber with 2% isoflurane-oxygen and moved onto a 37° C. heated platform with a nose cone. 26-gauge veterinary catheters were placed in each tail and the preloaded tubing was connected. Agents were injected over 30 sec, catheters were removed, and animals placed in the prepared Spectrum imaging chamber. Images were acquired beginning 10 min after injecting the agents. For SBK2-CCLE-IR800 and Scram-CCLE-IR800, images were acquired every 10 min for 60 min. For the ICG-labeled and TF8WS-labeled peptides, images were acquired at 10 min, 30 min, 1 h, 2 h, 4 h, 8 h, and 24 h. At the end of the imaging time, the animals were euthanized by CO₂ asphyxiation, and the tumor and other organs excised and imaged on the Spectrum.

### Data Analysis

Living Image analysis software was used for region of interest (ROI) analysis of the optical data. Images acquired for each time point consist of a fluorescent image superimposed on a photographic image. Binning was set to 1 and in vivo tumor ROIs were manually drawn based on contours visible in the black and white photograph for each timepoint. After outlining the tumor ROIs, fluorescence was measured with the built-in Spectrum function. For excised tumors, kidneys, spleen and liver, ROIs were drawn around the perimeter of the tumor or organ.

LivingImage software measures fluorescence intensity in units of radiant efficiency. For these analyses, average radiant efficiency measurements in (photons/s/cm²/steradian)/(μW/cm²) were used for further comparisons to account for differences in tumor and organ size. GraphPad Prism 9 was used for plots and statistical analyses. Where indicated, means for pairs of agents were compared using a two-tailed t-test with Welch's correction and statistical significance was assumed at p<0.05. Welch's correction was used to eliminate assumptions about variance between the groups being compared.

### Results

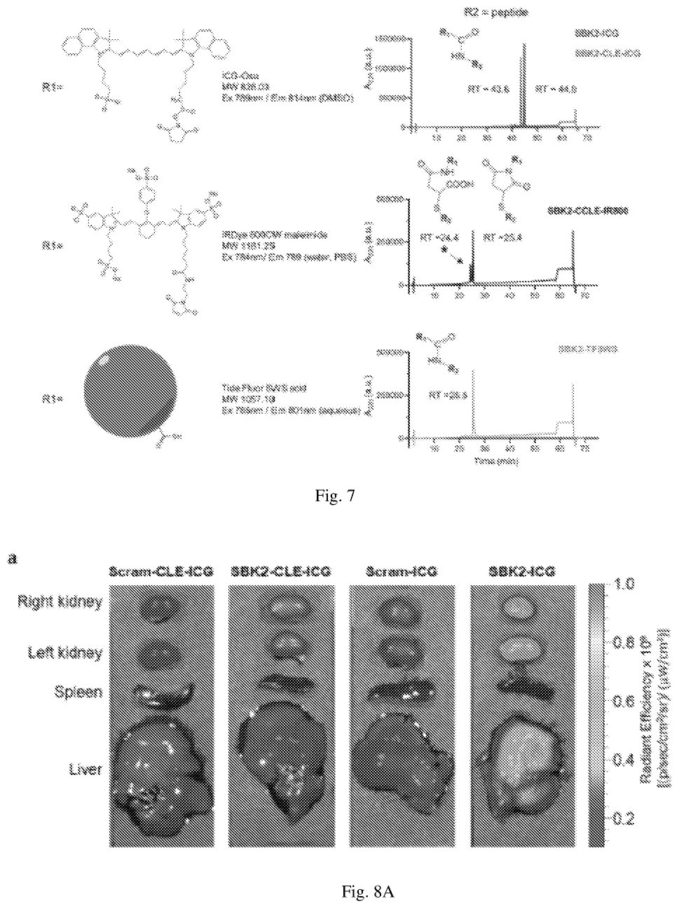

A series of near-infrared imaging agents targeted to the PTPμ biomarker, and appropriate control agents employing Scrambled peptides, were made by conjugating the NIR fluorophores ICG, IRDye® 800CW or Tide Fluor™ 8WS to the N-termini of the peptides. These agents are listed in Table 1. Additional structural information and characteristics are provided in Supplementary Table 1. ICG and Tide Fluor™ 8WS were conjugated to the N-terminal amine of resin-bound peptides such that an amide bond was formed between the fluorophore and the peptide (FIG. 7). Although conjugated in similar ways, differences in fluorophore hydrophobicity greatly influence the reverse phase (RP) HPLC retention times displayed by the ICG-labeled and TF8WS-labeled peptides as shown in FIG. 7. Both SBK2-ICG and SBK2-CLE-ICG require >45% acetonitrile/0.1% TFA to elute off the column and show RTs of 44.9 min and 43.6 min, respectively (FIG. 7).

TABLE 1
Near-infrared imaging agents targeted to the PTPu-biomarker. Fluorophores were
added to the N-terminus of specific (SBK2) and control (Scram) peptides.
The predicted pKa of all peptides used is 3.77

| | | | MW of |
|---|---|---|---|
| Name | Fluorophore | Peptide sequence | agent |
| SBK2-ICG | ICG | GEGDDFNWEQVNTLTKPTSD | 2966.27 |
| | | (SEQ ID NO: 5) | |
| Scram-ICG | ICG | GFTQPETGTDNDLWSVDNEK | 2966.27 |
| | | (SEQ ID NO: 48) | |
| SBK2-CLE- | ICG | GGSGGS.GEGDDFNWEQVNTLTKPTS | 3368.63 |
| ICG | | D(SEQ ID NO: 39) | |
| Scram-CLE- | ICG | GGSGGS.GFTQPETGTDNDLWSVDNE | 3368.63 |
| ICG | | K(SEQ ID NO: 49) | |
| SBK2-CCLE- | IRDye ® 800CW | C.GGSGGS.GEGDDFNWEQVNTLTKPT | 3881.11 |
| IR800* | | SD(SEQ ID NO: 50) | |
| Scram-CCLE- | IRDye ® 800CW | C.GGSGGS.GFTQPETGTDNDLWSVDN | 3881.11 |
| IR800* | | EK(SEQ ID NO: 51) | |
| SBK2-TF8WS | Tide Fluor ™ 8WS | GEGDDFNWEQVNTLTKPTSD | 3178 |
| | | (SEQ ID NO: 5) | |
| Scram-TF8WS | Tide Fluor ™ 8WS | GFTQPETGTDNDLWSVDNEK | 3178 |
| | | (SEQ ID NO: 48) | |

*MW of open succinimide ring form is 3899.12

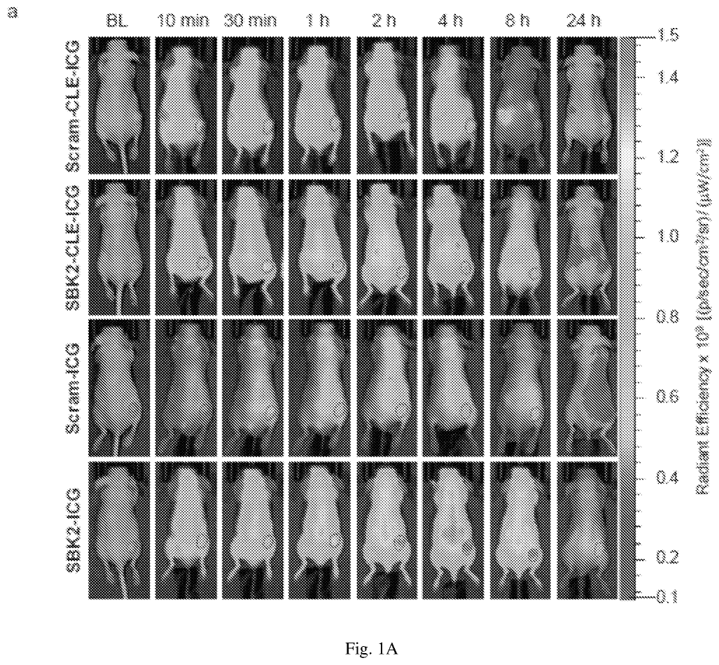

Interestingly, the six amino acid “CLE” insertion between ICG and SBK2 results in a retention time shorter by ˜1.3 min compared to SBK2-ICG (FIG. 1). Tide Fluor™ 8WS is described by its manufacturer as an alternative to IRDye® 800CW with similar hydrophilic properties but a broader pH compatibility. The more hydrophilic character of SBK2-TF8WS relative to the ICG-labeled peptides is apparent by its much shorter RT of 25.5 min.

A different strategy was employed for making agents with the hydrophilic, and low pH-sensitive IRDye® 800CW. Since the SBK2 and Scrambled peptides contain lysine, an additional cysteine residue was added to the N-terminus of each peptide and the dye was added in aqueous solution via a thiol-maleimide reaction with IRDye® 800CW maleimide (Table 1, FIG. 7). A closed succinimide ring is formed linking the dye to the peptide.

Similar to Tide Fluor™ 8WS, the hydrophilic nature of the IRDye® 800CW results in elution of the SBK2-CCLE-IR800 at around 25 min (FIG. 7). In assessing the IRDye® 800CW-labeled agents by RP-HPLC however, a second peak appeared when the SBK2-CCLE-IR800 and Scrambled-CCLE-IR800 stocks were warmed to room temperature. LC-MS/MS analysis was performed and confirmed the identity of the main peak as belonging the agents with the closed succinimide ring, while the second peak, representing around 30% of the material, and eluting slightly earlier than the main peak was identified as containing an open succinimide ring (FIG. 1). Conversion from a closed succinimide ring to a more stable open ring has been documented as occurring in other conjugates formed using thiol-maleimide reactions.

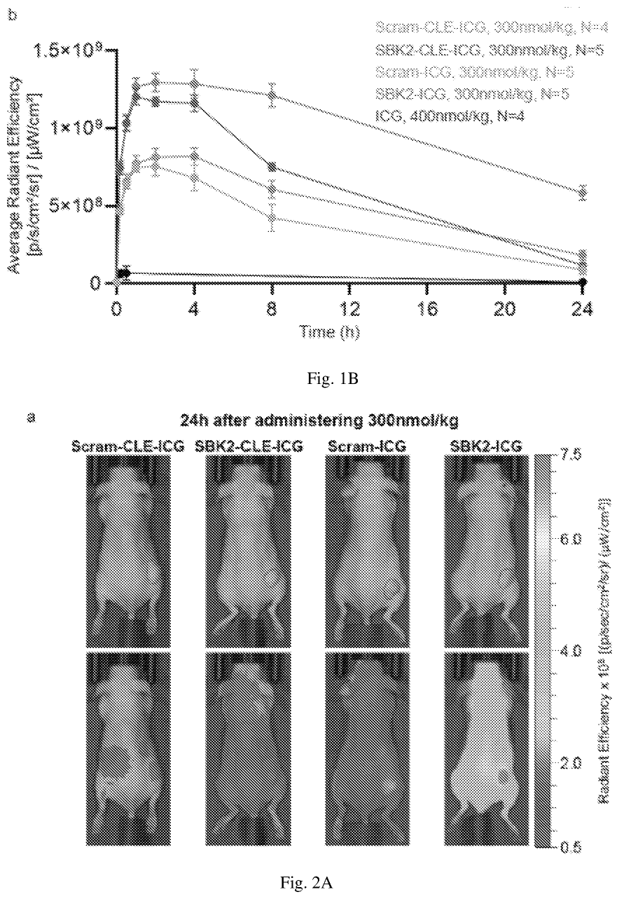

The ICG-labeled peptides were evaluated in vivo by injecting LN-229 glioma flank tumor-bearing nude mice with each of the four agents and acquiring images at various times out to 24 h. As shown in FIG. 1A, overall fluorescence rapidly increased following injection of the ICG agents at 300 nmol/kg and continued to increase for about 2 h. While fluorescence induced by the Scrambled control agents remained diffuse throughout the imaging period, the signal became much more pronounced specifically in the tumor region of animals treated with SBK2-CLE-ICG and SBK2-ICG. At 30 min, a fluorescent tumor is apparent in the animals treated with SBK2-CLE-ICG and remains highly fluorescent out to 4 hours (FIG. 1). At the 2 and 4 h time points, some fluorescence is also visible in the kidneys in mice treated with SBK2-CLE-ICG (FIG. 1A) as the agent clears. For mice treated with SBK2-ICG, similar in vivo tumor labeling is observed with slightly different kinetics. Specific fluorescence in the tumor region is visible at 30 min and becomes more pronounced by 1 h following SBK2-ICG administration and persists all the way out to 24 h (FIG. 1A, FIG. 2A). FIG. 1B shows average radiant efficiencies (mean±standard error) over time of in vivo tumor signal in mice administered 300 nmol/kg of each agent. The p-values obtained from unpaired t-tests between agents at each time point are summarized in Tables 2 and 3. SBK2-ICG produced significantly higher in vivo tumor fluorescence compared to either Scram-ICG or Scram-CLE-ICG from 10 min out to 24 h (FIG. 1B). Animals treated with SBK2-ICG also had significantly higher in vivo tumor fluorescence compared to animals treated with SBK2-CLE-ICG at 8 h and at 24 h (FIG. 1B). SBK2-CLE-ICG dosing led to significantly higher in vivo tumor signals relative to Scram-CLE-ICG, from 10 min out to 8 h, and relative to Scram-ICG, from 10 min out to 4 h (FIG. 1B). Injection of ICG dye at 400 nmol/kg (approximately 0.34 mg/mL), produced very little in vivo tumor fluorescence and cleared much more rapidly than any of the ICG-conjugated peptides (FIG. 1B). The peak ICG vivo tumor signal occurred by 10 min. In vivo tumor fluorescence was significantly higher in animals treated with ICG-labeled peptides compared to the base ICG dye except for the Scram-CLE-ICG at 24 h (FIG. 1B, Table 3).

Images and measurements acquired 24 h after dosing with the ICG-peptides are shown in FIG. 2. FIG. 2A displays the 24 h time point shown in FIG. 1A on a different scale, with the corresponding photos. Much more tumor-specific fluorescence is detected in the animal treated with 300 nmol/kg SBK2-ICG (FIG. 2A) compared to the animals injected with the other agents. FIG. 2B shows the 24 h in vivo tumor signals (mean±SEM) from mice treated with 300 nmol/kg or 400 nmol/kg of each agent or ICG at 400 mol/kg. Means were compared as described above, and the p-values are summarized in Tables 2-4. SBK2-ICG at 300 nmol/kg and at 400 nmol/kg produced significantly more tumor fluorescence at 24 h compared to all other ICG-conjugated peptides at the same dose (FIG. 2B, Tables 2 and 4). At 400 nmol/kg, SBK2-CLE-ICG produced significantly more in vivo tumor fluorescence compared to Scram-CLE-ICG (FIG. 2B). Scram-ICG, when used at 400 nmol/kg (N=6) appeared to clear more slowly than either of the CLE-containing agents and significantly higher in vivo tumor signals were detected in animals with this agent compared to either SBK2-CLE-ICG or Scram-CLE-ICG (FIG. 2B, Table 4). At 400 nmol/kg, all four ICG-peptides produced significantly higher in vivo tumor signals at 24 h compared to ICG dye only (FIG. 2B, Table 4).

TABLE 2
Summary of p-values from unpaired t-tests with Welch's
correction of in vivo tumor ROI means for 300 nmol/kg
doses of SBK2-ICG, Scram-CLE-ICG, SBK2-CLE-ICG, and
Scram- ICG. No significant differences were observed
between Scram-CLE-ICG and Scram-ICG at any time
SBK2-ICG, 300 nmol/kg, N = 5

vs.

| | Scram- | SBK2-CLE- | |
|---|---|---|---|
| | CLE-ICG, | ICG, | Scram-ICG, |
| | 300 nmol/kg, | 300 nmol/kg, | 300 nmol/kg, |
| | N = 4 | N = 5 | N = 5 |
| BL | ns | ns | ns |
| 10 min | <0.001 | ns | <0.001 |
| 30 min | <0.001 | ns | <0.001 |
| 1 h | <0.001 | ns | <0.001 |
| 2 h | <0.001 | ns | <0.001 |
| 4 h | 0.003 | ns | 0.007 |
| 8 h | <0.001 | 0.002 | <0.001 |
| 24 h | <0.001 | <0.001 | <0.001 |

SBK2-CLE-ICG, 300 nmol/kg, N = 5

vs

| | Scram- | |
|---|---|---|
| | CLE-ICG, | Scram-ICG, |
| | 300 nmol/kg, | 300 nmol/kg, |
| | N = 4 | N = 5 |
| BL | ns | ns |
| 10 min | <0.001 | <0.001 |
| 30 min | <0.001 | <0.001 |
| 1 h | <0.001 | <0.001 |
| 2 h | 0.002 | 0.001 |
| 4 h | 0.004 | 0.004 |
| 8 h | 0.029 | ns |
| 24 h | ns | ns |

TABLE 3
Summary of p-values from unpaired t-tests with Welch's
correction of in vivo tumor ROI means at available time
points for 400 nmol/kg ICG compared to 300 nmol/kg doses
of SBK2- ICG, Scram-CLE-ICG, SBK2-CLE-ICG, and Scram-ICG
ICG, 400 nmol/kg, N = 4

| | vs | | | |
|---|---|---|---|---|
| | Scram- CLE- | SBK2-CLE- | | |
| | ICG, | ICG, | Scram-ICG, | SBK2-ICG, |
| | 300 nmol/kg, | 300 nmol/kg, | 300 nmol/kg, | 300 nmol/kg, |
| | N = 4 | N = 5 | N = 5 | N = 5 |
| 10 min | <0.001 | <0.001 | <0.001 | <0.001 |
| 30 min | <0.001 | <0.001 | <0.001 | <0.001 |
| 24 h | ns | <0.001 | <0.001 | <0.001 |

TABLE 4
Summary of p-values from unpaired t-tests with Welch's correction
using ICG agents at 400 nmol/kg for in vivo tumor signal at 24 h

| | Scram- | SBK2- | | |
|---|---|---|---|---|
| | CLE-ICG, | CLE-ICG, | Scram-ICG, | SBK2-ICG, |
| | N = 6 | N = 7 | N = 6 | N = 8 |
| ICG, N = 5 | <0.001 | <0.001 | <0.001 | <0.001 |
| Scram-CLE- | | 0.003 | <0.001 | <0.001 |
| ICG, N = 5 | | | | |
| SBK2-CLE- | 0.003 | | 0.001 | <0.001 |
| ICG, N = 7 | | | | |
| Scram-ICG, | <0.001 | 0.001 | | <0.001 |
| N = 6 | | | | |
| SBK2-ICG, | <0.001 | <0.001 | <0.001 | |
| N = 8 | | | | |

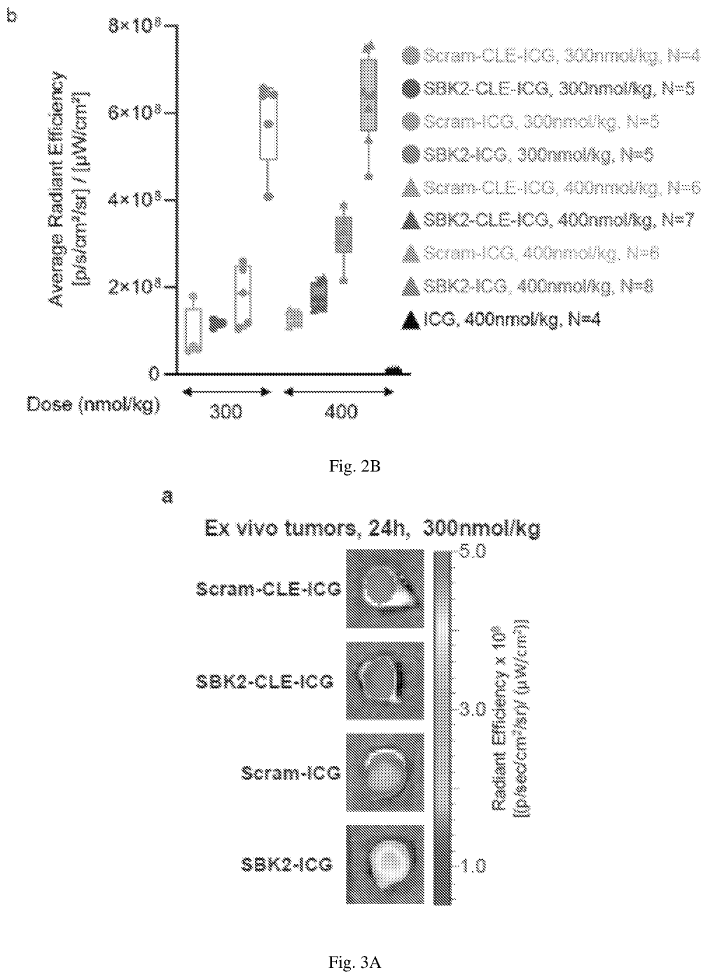

After acquiring 24 h images, mice were euthanized and the tumor, kidneys, and liver were excised along with spleen which served as a non-clearance organ comparison. Representative examples of ex vivo tumors taken at 24 h from animals injected with 300 nmol/kg are shown in FIG. 3A along with plots of the average radiant efficiencies (mean±SEM) of ex vivo tumors with agent at either 300 nmol/kg or 400 nmol/kg. FIG. 3A illustrates the substantially greater ex vivo tumor fluorescence present in mice treated with 300 nmol/kg SBK2-ICG. Ex vivo tumor fluorescence in mice treated with 300 nmol/kg SBK2-ICG was more than 3.5-fold higher than that in the other groups (FIG. 3B). At the higher dose of 400 nmol/kg, the fluorescence intensity ratios in mice given SBK2-ICG compared to the other agents were more modest. At 400 nmol/kg, the SBK2-ICG signal was about 1.8-fold higher than the Scam-ICG signal, suggesting more specificity was obtained at the lower dose. Compared to ICG only, tumor fluorescence from SBK2-ICG-treated mice varied from 52-fold higher (300 nmol/kg) to 70-fold higher (400 nmol/kg) demonstrating the value of including ICG-labeled Scrambled peptides as control agents (FIG. 3). Statistical comparisons are summarized in Table 5.

TABLE 5
Average radiant efficiency measurements from excised tumor and organs
in animals treated for 24 h with ICG-labeled agents at 300 nmol/kg
or ICG at 400 nmol/kg were compared using unpaired t-tests with
Welch's correction and the p-values obtained are summarized

| | Scram-CLE- | SBK2-CLE- | Scram-ICG, | SBK2-ICG, |
|---|---|---|---|---|
| | ICG, N = 4 | ICG, N = 5 | N = 5 | N = 5 |
| Tumor | | | | |
| ICG, 400 nmol/kg, N = 4 | 0.019 | 0.003 | 0.003 | <0.001 |
| Scram-CLE-ICG, N = 4 | | ns | ns | <0.001 |
| SBK2-CLE-ICG, N = 5 | ns | | ns | <0.001 |
| Scram-ICG, N = 5 | ns | ns | | <0.001 |
| SBK2-ICG, N = 5 | <0.001 | <0.001 | <0.001 | |
| Kidney | | | | |
| ICG, 400 nmol/kg, N = 4 | 0.008 | <0.001 | 0.003 | <0.001 |
| Scram-CLE-ICG, N = 4 | | 0.034 | ns | <0.001 |
| SBK2-CLE-ICG, N = 5 | 0.034 | | ns | <0.001 |
| Scram-ICG, N = 5 | ns | ns | | <0.001 |
| SBK2-ICG, N = 5 | <0.001 | <0.001 | <0.001 | |
| Spleen | | | | |
| ICG, 400 nmol/kg, N = 4 | <0.001 | <0.001 | 0.004 | <0.001 |
| Scram-CLE-ICG, N = 4 | | 0.015 | ns | <0.001 |
| SBK2-CLE-ICG, N = 5 | 0.015 | | ns | <0.001 |
| Scram-ICG, N = 5 | ns | ns | | <0.001 |
| SBK2-ICG, N = 5 | <0.001 | <0.001 | <0.001 | |
| Liver | | | | |
| ICG, 400 nmol/kg, N = 4 | <0.001 | 0.004 | 0.013 | <0.001 |
| Scram-CLE-ICG, N = 4 | | ns | ns | <0.001 |
| SBK2-CLE-ICG, N = 5 | ns | | ns | <0.001 |
| Scram-ICG, N = 5 | ns | ns | | <0.001 |
| SBK2-ICG, N = 5 | <0.001 | <0.001 | <0.001 | |

Tumors from SBK2-ICG-treated mice treated at 300 nmol/kg and 400 nmol/kg were significantly higher than the tumors excised from mice treated with the other agents at the same dose or 400 nmol/kg ICG (FIG. 3). At 300 nmol/kg, no significant differences were detected between the other agents. At 400 nmol/kg, tumors excised from mice treated with SBK2-CLE-ICG and Scram-ICG were both significantly more fluorescent than tumors excised from mice treated with Scram-CLE-ICG.

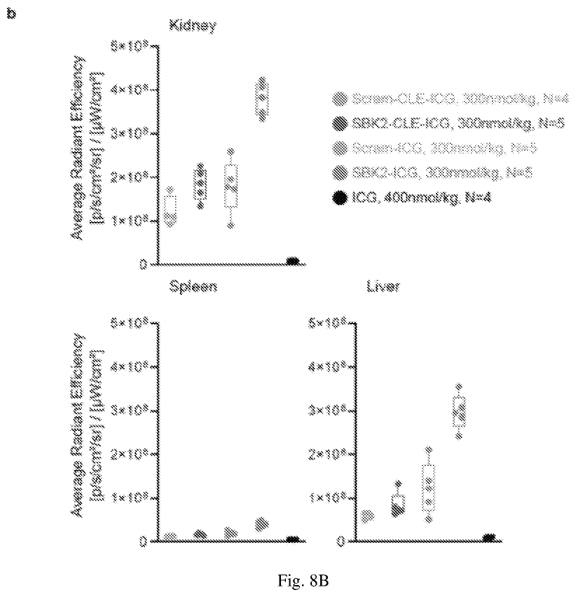

Clearance of ICG dye from the body occurs primarily through the liver. As shown in FIG. 8, fluorescence was detected to some extent in excised kidney and liver from mice treated with ICG-labeled peptides with little residual fluorescence detected in the spleen. Significantly higher amounts of fluorescence were detected in kidney, liver and spleen of the mice treated with SBK2-ICG compared to the other mice which is likely related to the overall slower clearance rate observed in these animals (FIG. 8, Table 5).

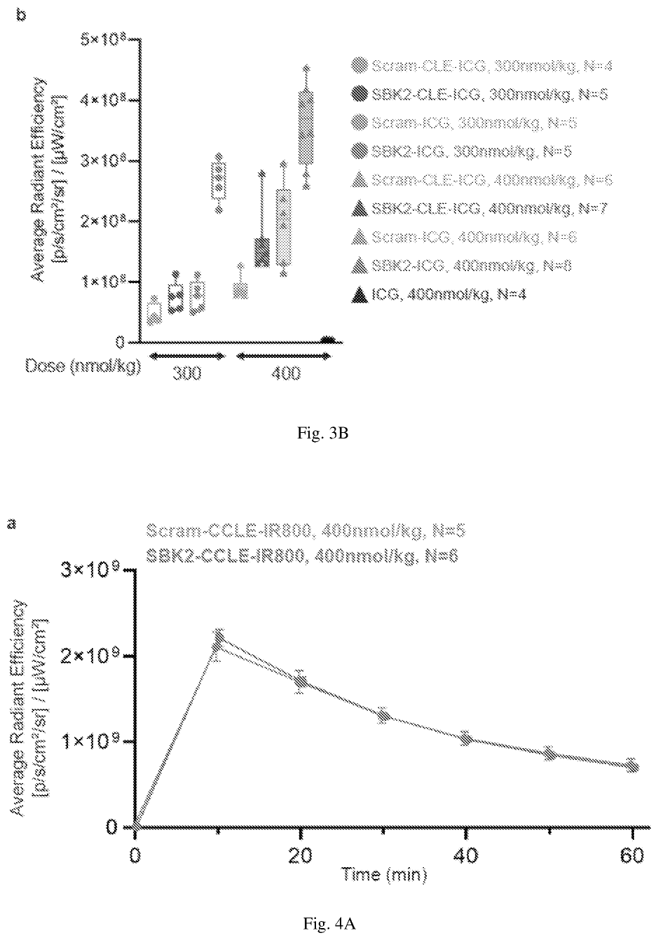

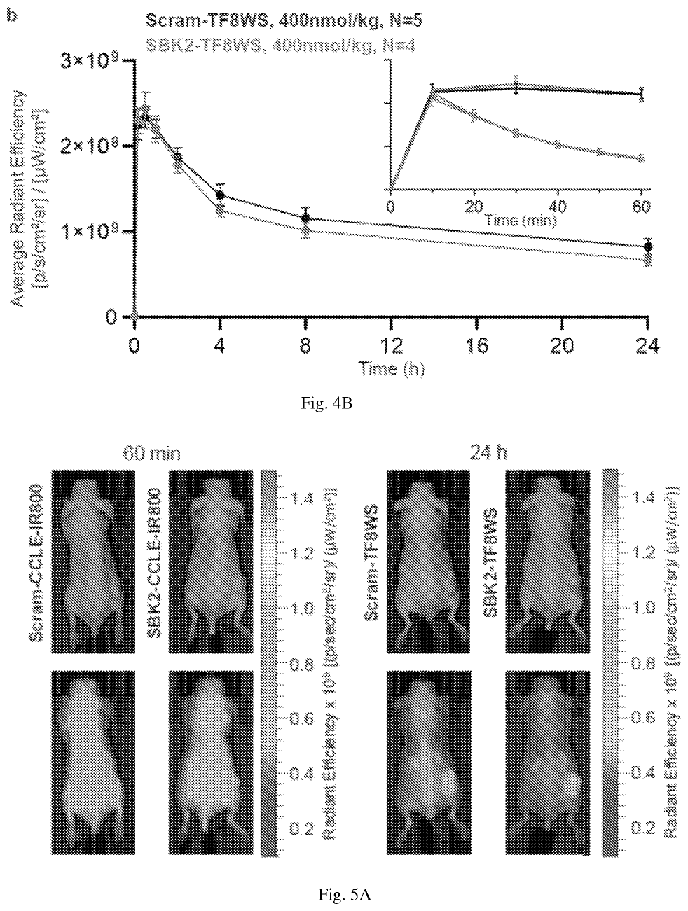

In addition to the ICG-labeled peptides, peptides conjugated to the more hydrophilic dyes IRDye® 800CW and Tide Fluor™ 8WS were studied for their ability to specifically label LN-229 flank tumors in nude mice. FIG. 5 shows time course of in vivo tumor fluorescence (mean±SEM) in mice given 400 nmol/kg of SBK2-CCLE-IR800 or Scram-CCLE-IR800 (FIG. 4A), and SBK2-TF8WS or Scram-TF8WS (FIG. 4B). In contrast to the ICG-conjugated peptides, no specific tumor labeling was observed using either SBK2-CCLE-IR800 or SBK2-TF8WS. There was very high non-specific overall body fluorescence with the IR800 agents (FIG. 4A). The inset panel in FIG. 4B shows the data obtained from the mice administered the TF8WS-conjugated peptides plotted on the same scale used in FIG. 4A. All four agents were injected at 400 nmol/kg and similar levels of peak in vivo tumor fluorescence were observed 10 min following injection. Although both fluorophores are characterized as hydrophilic, and similar RT were obtained by RP-HPLC, different clearance characteristics were evident between the two classes of agents. The IRDye® 800CW-labeled peptides cleared rapidly and 60 min was determined to be an appropriate imaging endpoint. On the other hand, the TF8WS-conjugated agents peaked quickly but cleared more slowly than the IR800-agents. As seen in the inset plot, in vivo tumor fluorescence for both SBK2-TF8WS and Scram-TF8WS is relatively stable for the first 60 min and then gradually begins clearing.

Representative mice dosed with 400 nmol/kg Scram-CCLE-IR800 and SBK2-CCLE-IR800 are shown 60 min following injection in FIG. 5A (left) along with animals 24 h after receiving 400 nmol/kg of Scram-TF8WS or SBK2-TF8WS (FIG. 5A, right). In contrast to the IR800-conjugated peptides, both TF8WS-labeled peptides resulted in a strong signal localized to the tumor indicating a strong EPR effect. Plots of in vivo tumor fluorescence (mean±SEM) at 60 min (IR800-agents), and at 24 h (TF8Ws-agents) are shown in FIG. 5B. No statistical differences were detected between either the IR800-labeled agents or between the TF8WS-labeled agents.

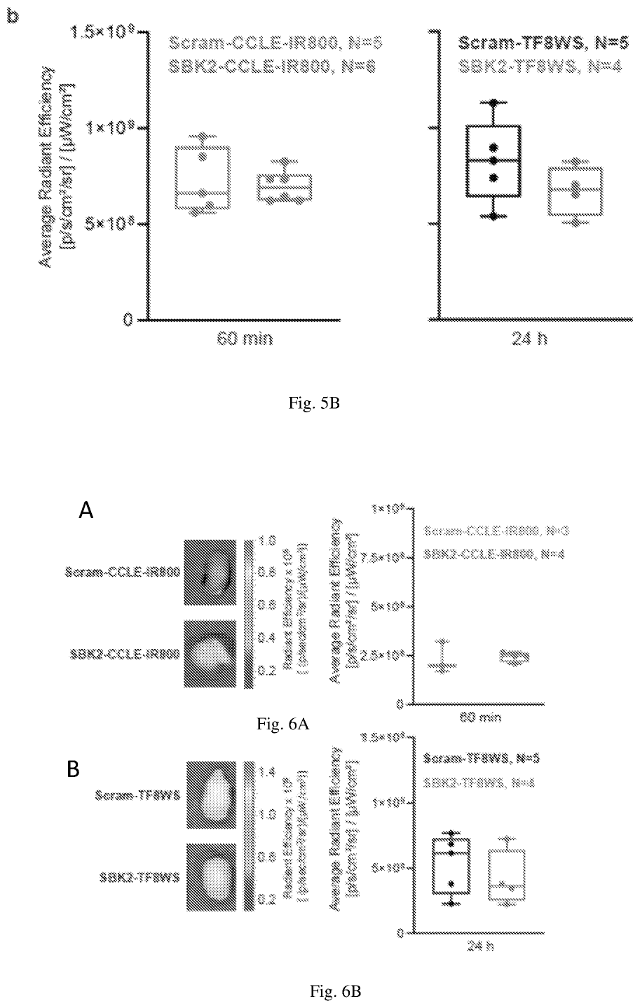

Representative excised tumors from mice treated with each type of agent are shown in FIG. 6 along with corresponding plots of average radiant efficiency values (mean±SE). No statistical differences were observed between the ex vivo tumor signals obtained from mice given either type of IR800-conjugated agent or between those from the mice injected with the Tide Fluor™ 8WS-labeled agents.

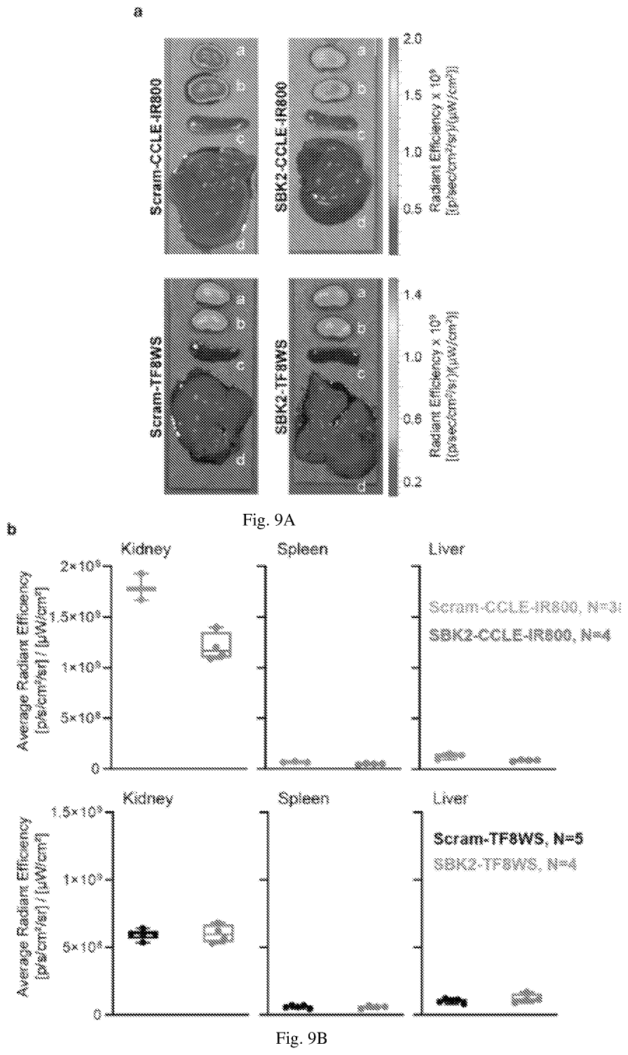

In addition to tumor, kidneys, spleen and liver were excised from the animals given the IR800-labeled and TF8WS-conjugated agents. Representative examples of these organs are shown in FIG. 9A with plots of the average radiant efficiency (mean±SEM) for each organ type in FIG. 9B. The highest fluorescent signals were detected in the kidneys. Kidneys from SBK2-CCLE-IR800-treated animals were significantly less fluorescent than those from animals treated with Scram-CCLE-IR800 (FIG. 9B). No significant difference in liver fluorescence was observed, but spleen fluorescence in the mice treated with Scram-CCLE-IR800, though low, was significantly higher than in mice dosed with SBK2-CCLE-IR800 (FIG. 9B). For animals treated with SBK2-TF8WS and Scram-TF8WS, the highest fluorescent signals were detected in kidney with low amounts measured in the spleens and livers. No significant differences between SBK2-TF8WS and Scram-TF8WS were observed in any of these three organs.

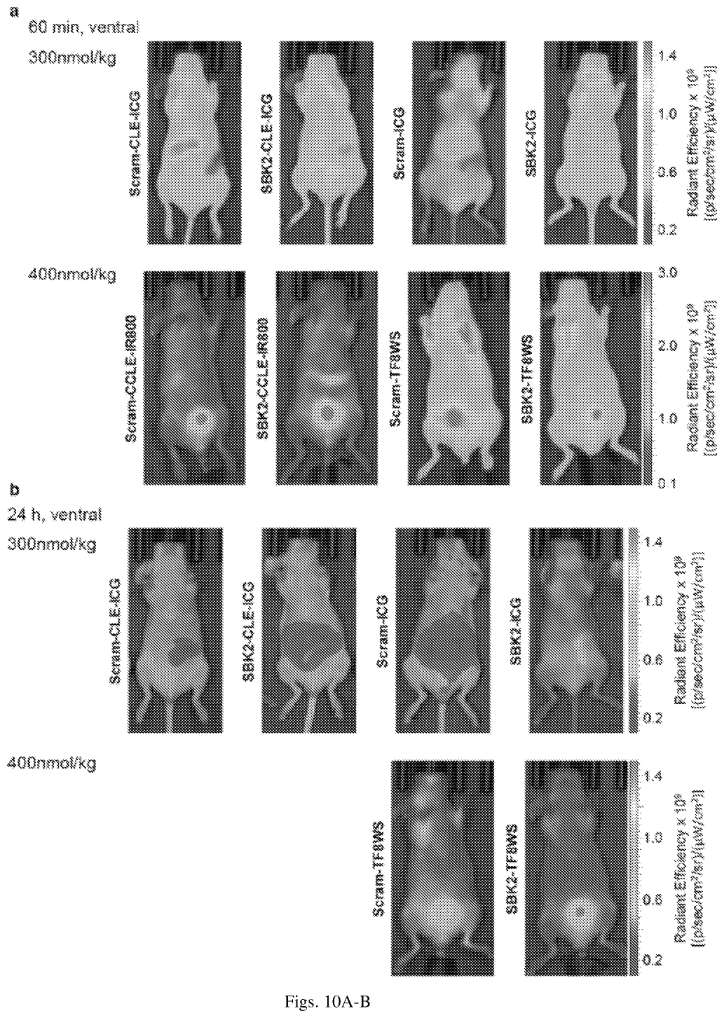

Ventral images illustrate the biodistribution of agents as they clear through the bladder. Ventral images were acquired at 1 h for all sets of animals and at 24 h for those treated with the ICG-labeled and TF8WS-labeled agents (FIG. 10). For mice treated with ICG-conjugated peptides, more diffuse fluorescence throughout the abdomen is visible. In contrast, the mice treated with the more hydrophilic IRDye® 800CW- and Tide Fluor™ 8WS-conjugated peptides display intense fluorescence localized to the bladder suggesting a key role for renal clearance of these agents.

In recent years, FIGS with non-targeted agents has benefitted many patients and extended lives by enabling resection of the bulk of the tumor. However, tumor recurrence is a problem especially at the edge of the resection. Recent efforts have focused on creating molecularly targeted agents that will enhance the ability of the surgeon to delineate tumor margins from surrounding normal tissue. For small molecule imaging agents, identifying the ideal attributes of targeting moiety, linker and fluorophore combination is an ongoing process as how these components work together to create a novel single agent that works in vivo remains unpredictable. Our studies contribute to this active area of research in two important areas. First, two molecularly targeted NIR-imaging agents, SBK2-ICG and SBK2-CLE-ICG, are described that show specific in vivo tumor labeling relative to controls in a mouse model of human glioblastoma. The tumor labeling achieved with SBK2-ICG shortly after injection and out to at least 24 h is particularly compelling especially for FIGS. Second, by employing three different fluorophores and a linker, these studies add critical insights to the growing effort to understand how fluorophores and linkers contribute to the in vivo behavior of an agent.

ICG was first approved for clinical use in 1959 and continues to serve a vital function in many imaging applications. New insights into the use of this fluorophore continue today. Recent work has revealed that in addition to fluorescence in the NIR-I range, ICG fluorescence can also be detected in the NIR-II (1000-1700 nm) range suggesting an opportunity for deeper tissue imaging. In addition, surgeons have recently explored administration of high dose (5 mg/kg) ICG with FIGS at 24 h in a technique called “Second Window ICG” (SWIG) to take advantage of the EPR effect. For comparison, the 300 nmol/kg=0.89 mg/kg dose of SBK2-ICG used in these studies equates to a Human Equivalent Dose of around 0.07 mg/kg (or 70 μg/kg). By combining the SBK2 targeting peptide with ICG, specific tumor labeling occurred rapidly and persisted out to 24 h at a smaller dose than that used for SWIG.

For these studies, agents utilizing the hydrophilic IRDye® 800CW and Tide Fluor™ 8WS were also examined. IRDye® 800CW in combination with different antibody-based targeting moieties has shown great promise so the lack of any detectable specificity by SBK2-CCLE-IR800 was unexpected. Further, the clearance by both the SBK2-CCLE-ICG and Scram-CCLE-ICG via the renal system occurred more rapidly than expected. Injection of IRDye® 800CW dye only resulted in more overall fluorescence throughout the body than achieved by the IRDye® 800CW-labeled peptides at the same dose and cleared more slowly than the peptide-containing agents (data not shown). While unexpected, these results reinforce the notion that fluorophore characteristics greatly influence the biodistribution of small molecule imaging agents. In contrast to the ICG-labeled peptides, which exhibited much slower clearance than ICG alone, it is not readily apparent why the IRDye® 800CW-labeled peptides exhibited an increased clearance rate relative to the fluorophore on its own. The hydrophilic SBK2-TF8WS peptides demonstrated no specificity for the PTPμ-biomarker expressing tumors either, indicating hydrophilic dyes may not be the best option for use with these targeting peptides. While not specific for PTPμ, administration of both SBK2-TF8WS and Scram-TF8WS led to a high tumor-specific signal at 24 h.

These data demonstrate specific in vivo labeling of human glioma in an appropriate timeframe using PTPμ-targeting peptides conjugated to the NIR fluorophore ICG. SBK2-ICG achieved significantly higher tumor labeling than control agents within 10 min of administration and a tumor-specific signal persisted for at least 24 h. Similarly, SBK2-CLE-ICG, with an additional six amino acid linker, demonstrated specific in vivo tumor labeling but cleared more rapidly. In contrast, more hydrophilic NIR agents utilizing the IRDye® 800CW and Tide Fluor™ 8WS fluorophores showed no specificity. Injection of peptides conjugated to IRDye® 800CW resulted in rapid, diffuse fluorescence which had cleared extensively by 60 min through the kidneys. Tide Fluor™ 8WS-labeled SBK2 and Scram peptides both produced highly fluorescent non-specific tumor signals which lasted out to at least 24 h. The properties of SBK2-ICG make an ideal agent for FIGS of glioblastoma and we are currently working toward clinical trial with this agent.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

1. A near-infrared imaging agent comprising:
a targeting peptide that specifically binds to and/or complexes with a proteolytically cleaved extracellular fragment of an immunoglobulin (Ig) superfamily cell adhesion molecule that is expressed by a cancer cell or another cell in the cancer cell microenvironment;
an optional spacer directly linked to the targeting peptide; and
a near-infrared fluorophore that is directly or indirectly linked to the targeting peptide or optional spacer via a natural or non-natural linkage.

2. The near-infrared imaging agent of claim 1, wherein the agent administered to a subject has a signal to background ratio (SBR) upon fluorescent imaging effective to delineate the cancer cell or another cell in the cancer cell microenvironment from surrounding tissue.

3. The near-infrared imaging agent of claim 1, wherein the near-infrared fluorophore is hydrophobic or lipophilic.

4. The near-infrared imaging agent of claim 1, wherein the non-natural linkage is not susceptible to proteolytic cleavage.

5. The near-infrared imaging agent of claim 1, wherein the non-natural linkage includes an amide that links the targeting peptide or optional spacer to the near-infrared fluorophore.

6. The near-infrared imaging agent of claim 1, having formula: 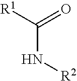
or a pharmaceutically acceptable salt thereof;
wherein R¹ is the near-infrared fluorophore; and
R² includes the targeting peptide and optional spacer.

7. The near-infrared imaging agent of claim 1, wherein near-infrared fluorophore includes at least one of a cyanine near-infrared fluorophore having a fluorescence in the first near infrared region or second near infrared region.

8. The near-infrared imaging agent of claim 7, wherein the cyanine near-infrared fluorophore is a heptamethine cyanine near-infrared fluorophore.

9. The near-infrared imaging agent of claim 1, wherein near-infrared fluorophore includes at least one of indocyanine green (ICG) or ICG-Osu.

10. The near-infrared imaging agent of claim 1, having formula: 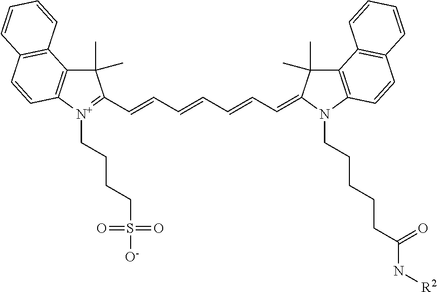
or a pharmaceutically acceptable salt thereof;
wherein R² includes the targeting peptide and optional spacer.

11-18. (canceled)

19. The near-infrared imaging agent of claim 10, wherein the spacer comprises consists of the amino acid sequence of at least one of (GS)a, (GGS)b, or (GGGS)c, or (GGGGS)d and wherein a, b, c, and d are each independently 2, 3, 4, 5, or 6.

20-31. (canceled)

32. A compound of formula: 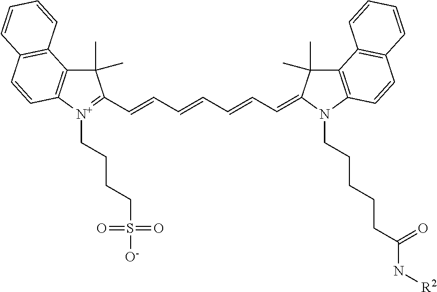
or a pharmaceutically acceptable salt thereof;
wherein R² includes a targeting peptide that specifically binds to and/or complexes with a proteolytically cleaved extracellular fragment of an immunoglobulin (lg) superfamily cell adhesion molecule that is expressed by a cancer cell or another cell in the cancer cell microenvironment and an optional spacer directly linked to the targeting peptide.

33. The compound of claim 32, wherein R² consists of the targeting peptide.

34. The compound of claim 32, wherein R² consists of the targeting peptide linked to a spacer and wherein the spacer separates the amide from the targeting peptide.

35. The compound of claim 34, wherein the spacer has a length and structure effective to at least maintain or preserve binding affinity of the linked targeting peptide to the proteolytically cleaved extracellular fragment.

36-40. (canceled)

41. The compound of claim 32, wherein spacer comprises the amino acid sequence of at least one of (GS)a, (GGS)b, or (GGGS)c, or (GGGGS)d and wherein a, b, c, and d are each independently 2, 3, 4, 5, or 6.

42. The near-infrared imaging agent of claim 10, wherein R² includes a targeting peptide having an amino acid sequence selected from SEQ ID NO: 4, SEQ ID NO: 5, SEQ ID NO: 6, or SEQ ID NO: 7.

43. The near-infrared imaging agent of claim 10, wherein R² consists of an amino acid sequence selected from SEQ ID NO: 5 or SEQ ID NO: 39.

44. The compound of claim 32, wherein R² includes a targeting peptide having an amino acid sequence selected from SEQ ID NO: 4, SEQ ID NO: 5, SEQ ID NO: 6, or SEQ ID NO: 7.

45. The compound of claim 32, wherein R² consists of an amino acid sequence selected from SEQ ID NO: 5 or SEQ ID NO: 39.
